SEQUENCE LISTING (17 sequences)

SEQ ID NO: 1 - DNA, 45 bp, Artificial Sequence
  source: /note=“Description of Artificial Sequence Synthetic primer”
ctggtgaaaacaccgcagcatgtcgcacgagtgagccctgggcgg

SEQ ID NO: 2 - DNA, 24 bp, Artificial Sequence
  source: /note=“Description of Artificial Sequence Synthetic primer”
gcatggtattctttctcttccgca

SEQ ID NO: 3 - DNA, 45 bp, Artificial Sequence
  source: /note=“Description of Artificial Sequence Synthetic primer”
agacaactgttcaaactgatgggaaaacacaatcatctatttctc

SEQ ID NO: 4 - DNA, 24 bp, Artificial Sequence
  source: /note=“Description of Artificial Sequence Synthetic primer”
agacaactgttcaaactgatggga

SEQ ID NO: 5 - DNA, 24 bp, Artificial Sequence
  source: /note=“Description of Artificial Sequence Synthetic primer”
ctggtgaaaacaccgcagcatgtc

SEQ ID NO: 6 - DNA, 24 bp, Artificial Sequence
  source: /note=“Description of Artificial Sequence Synthetic primer”
agacaactgttcaaactgatggga

SEQ ID NO: 7 - DNA, 22 bp, Artificial Sequence
  source: /note=“Description of Artificial Sequence Synthetic oligonucleotide”
ggagcaaaatagcaatgaggta

SEQ ID NO: 8 - DNA, 45 bp, Artificial Sequence
  source: /note=“Description of Artificial Sequence Synthetic primer”
ctggtgaaaacaccgcagcatgtcgcccgagtgagccctgggcag

SEQ ID NO: 9 - DNA, 45 bp, Artificial Sequence
  source: /note=“Description of Artificial Sequence Synthetic primer”
gaggaatgaaacggaggaagacgtacgtattctttctcttccgca

SEQ ID NO: 10 - DNA, 125 bp, Artificial Sequence
  source: /note=“Description of Artificial Sequence Synthetic polynucleotide”
ctaaagccacctccttactttgcctccttctgcatggtattctttctcttccgcatgcaccagtttggcctgcccaaaatctgtgatcttgacatgctgcggtgttttcaccagtacgttcctgg

SEQ ID NO: 11 - DNA, 125 bp, Artificial Sequence
  source: /note=“Description of Artificial Sequence Synthetic polynucleotide”
ctaaagccacctccttactttgcctccttctgcatggtattctttctcttccgcatgcaccagttcggcccgcccaaaatctgtgatcttgacatgctgcggtgttttcaccagtacgttcctgg

SEQ ID NO: 12 - DNA, 30 bp, Artificial Sequence
  source: /note=“Description of Artificial Sequence Synthetic oligonucleotide”
cagctgcactgggaagggtgcagtctgacc

SEQ ID NO: 13 - DNA, 30 bp, Artificial Sequence
  source: /note=“Description of Artificial Sequence Synthetic oligonucleotide”
ggtcagactgcacccttcccagtgcagctg

SEQ ID NO: 14 - DNA, 125 bp, Artificial Sequence
  source: /note=“Description of Artificial Sequence Synthetic polynucleotide”
ctaaagccacctccttactttgcctccttctgcacggtattctttctcttccgcatgcaccagtttggcctgcccaaaatctgtgatcttgacatgctgcggtgttttcaccagtacgttcctgg

SEQ ID NO: 15 - DNA, 15 bp, Artificial Sequence
  source: /note=“Description of Artificial Sequence Synthetic probe”
ccaaactggtgcggg

SEQ ID NO: 16 - DNA, 11 bp, Artificial Sequence
  source: /note=“Description of Artificial Sequence Synthetic oligonucleotide”
gaataatatag

SEQ ID NO: 17 - DNA, 11 bp, Artificial Sequence
  source: /note=“Description of Artificial Sequence Synthetic oligonucleotide”
ctatattattc

(19) United States
(12) Patent Application Publication
(10) Pub. No.: US 2016/0355881 A1
(43) Pub. Date: Dec. 8, 2016

(54) DNA Methylation Analysis

(71) Applicant: Brandeis University, Waltham, MA (US)

(72) Inventors: Lawrence J. Wangh, Auburndale, MA (US); Jesus A Sanchez, Framingham, MA (US)

(21) Appl. No.: 14/913,862

(22) PCT Filed: Jul. 24, 2014
(86) PCT No.: PCT/US14/48036

§ 371 Date: Feb. 23, 2016

Related U.S. Application Data
(60) Provisional application No. 61/857,941, filed on Jul. 24, 2013.

(51) Int. Cl. C12Q 1/68

(52) U.S. Cl. CPC C12Q 1/6858

(57) ABSTRACT

Disclosed herein are methods for determining whether one or more CpG dinucleotides are methylated using Linear After the Exponential Polymerase Chain Reaction (“LATE-PCR”) or Linear-Expo-Linear Polymerase Chain Reaction (“LEL-PCR”).

### RELATED APPLICATIONS

This application claims the benefit of priority to U.S. Provisional Patent Application Ser. No. 61/857,941, filed Jul. 24, 2013; the contents of which are hereby incorporated by reference.

### BACKGROUND

DNA methylation in eukaryotes is a key regulator of gene expression during both normal and abnormal cellular growth and development. This regulatory mechanism is dictated by the gain or loss of a methyl-group on the 5-position of a cytosine in a CpG dinucleotide. In general, methylated CpGs are present in heterochromatin and correlate with a lack of gene expression, whereas unmethylated CpGs are present in euchromatin and correlate with actively expressed genes.

Methyl groups are covalently attached to nucleotides by enzymes known as methyltransferases. In the case of 5-methyl-cytosine, the targeted nucleotide is always a CpG dinucleotide, and often the dinucleotide is part of a tetranucleotide having one of two possible configurations: CpCpGpGp or CpGpCpGp. Because double-stranded DNA is a self-complementary anti-parallel double helix, these CpG sequences are palindromic, meaning that their complementary anti-parallel strands in double-stranded DNA have the same sequence in the opposite direction. This, in turn, means that a particular CpG/GpC can either be unmethylated, hemimethylated (m)CpG/GpC, or fully methylated (m)CpG/GpC(m).

CpG dinucleotides are underrepresented throughout the genome; DNA sequences upstream of transcribed genes frequently contain many CpG dinucleotides. Such regions are referred to as CpG islands. While, in many cases, an entire region of the genome will share the same CpG methylation state, there are some genes that display subtle patterns of methylated and unmethylated CpG dinucleotides in their transcription control regions and changes in such patterns of methylation and demethylation are indicative of changes in gene activity.

Abnormal patterns of methylation and demethylation are observed in several epigenetic syndromes. Such syndromes typically involve the misregulation of many genes, many of which display abnormal methylation patterns. These syndromes can arise due to non-disjunction during meiosis, congenital chromosomal errors, trauma, stress, exposure to a toxin such as a heavy metal or heat, or even alterations in hormone levels or diet.

Alteration in methylation patterns also occur in cancer. For instance, breast cancer often features epigenetic changes due, in part, because the Brca1 gene product is responsible for regulating certain methyltransferases A decrease in the amount of the Brca1 protein, a change in its structure due to a mutation, or an alteration in the pattern of methylation of the Brca1 gene itself can result in a reduction in genome wide levels of DNA methylation.

In light of the foregoing, there is a great need for improved methods of determining the methylation state of a gene or a region of the genome.

### SUMMARY

In certain aspects, disclosed herein are methods using Linear After the Exponential Polymerase Chain Reaction (“LATE-PCR”) and Linear-Expo-Linear Polymerase Chain Reaction (“LEL-PCR”) to amplify a DNA target in a sequence specific manner and, in the case of DNA which may or may not be methylated, to determine the methylation status of one or more CpG dinucleotides. In some embodiments, Temperature Imprecise Polymerase Chain Reaction (“TI-PCR”) is used to perform the LATE-PCR or LEL-PCR amplification. In some embodiments, one or more SuperSelective primers are used during the LATE-PCR or LEL-PCR amplification. In some embodiments the CpG dinucleotide is located in a target region (e.g., a region encompassing a CpG island) of a DNA molecule.

In some embodiments the method includes the step of contacting the DNA molecule with a solution comprising bisulfite under conditions such that a majority of unmethylated cytosine residues in the DNA molecule are converted to uracil (e.g., greater than 90%) and a majority of methylated cytosine residues in the DNA molecule remain cytosine (e.g., greater than 90%). In some embodiments the DNA molecule is fragmented prior to bisulfite conversion. In some embodiments the DNA is fragmented using a restriction enzyme that cuts the DNA molecule outside of the region that is amplified. In some embodiments the method includes the step of amplifying the target region of the bisulfite-treated DNA molecule using LATE-PCR or LEL-PCR to generate an amplification product.

In some embodiments the method includes the step of purifying the DNA molecule prior to bisulfite conversion such that it is substantially protein-free. In some embodiments the purification step is performed using a proteinase K lysis solution containing 10 mM Tris-Cl pH 8.3, 5 μM SDS, and 0.1 mg/ml Proteinase K, or by adding a chaotropic salt.

In some embodiments the steps of purifying the DNA, bisulfite conversion, and/or amplification are carried out in a single tube. In some embodiments the steps are carried out by adding the appropriate reagents (e.g., reagents described herein) in sequence to the same tube through a series of steps that increase the volume of the reaction at each successive step sufficiently to dilute out possible interfering reactants from a previous step.

In some embodiments the method includes the step of determining whether the CpG dinucleotide was converted to uracil during a bisulfite conversion process. In certain embodiments, this step includes hybridizing the amplification product to at least one probe. In some embodiments the probe is a Low-Tm or Superlow-Tm probe that hybridizes to the amplification product at a position occupied by a CpG dinucleotide prior to bisulfite conversion in a manner that reflects the CpG dinucleotide's original methylation status. In some embodiments the probe is one member of a Lights-On/Lights-Off probe pair that hybridizes to the amplification product at the position originally occupied by the CpG dinucleotide. In some embodiments the probe is a Lights-Off only probe that hybridizes to the amplification product at the position occupied by a CpG dinucleotide prior to bisulfite conversion in a manner that reflects the CpG dinucleotide's original methylation status. In some embodiments the step includes sequencing at least part of the amplification product. In some embodiments the sequencing is performed using chain termination sequencing, sequencing by ligation, sequencing by synthesis, pyrosequencing, ion semiconductor sequencing, single-molecule real-time sequencing, dilute-‘n’-go sequencing and/or 454 sequencing. In some embodiments, Temperature Dependent Reagents are used in the LATE-PCR amplification reaction.

In some embodiments the original methylation status of more than one CpG dinucleotide located in the target region is detected. In some embodiments, the original methylation status of more than one CpG dinucleotide located in the target region is detected using more than one probe. In some embodiments the probes are two or more Low-Tm or Superlow-Tm probes that hybridize to the amplification product at the positions occupied by the two or more CpG dinucleotides prior to bisulfite conversion in a manner that reflects their original methylation status. In some embodiments the probe is one member of a set of Lights-On/Lights-Off probe pairs that hybridizes to the amplification product at the position of the two or more CpG dinucleotides. In some embodiments the probes are two or more Lights-Off Only probes that hybridize to the amplification product at positions occupied by the two or more CpG dinucleotides prior to bisulfite conversion in a manner that reflects their original methylation status. In certain embodiments the method includes the step of hybridizing the amplification product to a first set of probes and a second set of probes tagged with the same fluorescent label. In some embodiments the first set of probes and the second set of probes are tagged with different fluorescent labels.

In some embodiments the method of detecting the methylation status of a CpG dinucleotide located in a target region comprises the steps of: a) dividing a genomic DNA sample into a control genomic DNA solution containing a control DNA molecule containing the target region from a test genomic DNA solution containing a test DNA molecule containing the target region; b) contacting the test DNA molecule with a solution comprising bisulfite under conditions such that a majority of unmethylated cytosine residues in the DNA molecule are converted to uracil and a majority of methylated cytosine residues in the DNA molecule remain cytosine; c) amplifying the target region of the test DNA molecule using LATE-PCR using a first primer set complementary to bisulfite treated DNA to generate a first amplification product; or using LEL-PCR using a single first limiting primer that is complementary to bisulfite treated DNA to achieve linear accumulation of a first amplification product, followed by hybridization of a reverse primer to achieve exponential amplification of the product strands, followed by linear amplification of one product strand when the limiting primer is used up; d) amplifying the target region of the control DNA molecule (e.g., a control DNA molecule not treated with sodium bisulfite) using LATE-PCR or LEL-PCR using a secondary primer complementary to native DNA to generate a second amplification product; e) incubating the first amplification product with a first set of probes at different temperatures to generate a first fluorescent signature; f) incubating the second amplification product with a second set of probes to generate a second fluorescent signature; and g) comparing the first fluorescent signature with the second fluorescent signature to determine whether the CpG dinucleotide was methylated in the original genomic DNA sample. In some embodiments the first set of probes are detectably labeled with a first fluorescent label and the second set of probes are detectably labeled with a second fluorescent label. In some embodiments the first fluorescent label emits light at a different wavelength than the second fluorescent label. In some embodiments the comparing step includes a ratio analysis of the emission at the first wavelength and emission at the second wavelength at each temperature, in, for example, one degree centigrade intervals, from five degrees below the lowest temperature at which the maximum possible probes are bound to any target to five degrees above the temperature at which the highest temperature at which any probe is bound to any target. In some embodiments, the Differential Strand Separation at Critical Temperature (DISSECT) approach is used to determine the methylation status of the CpG dinucleotide located in the target region.

In some embodiments, the aforementioned control DNA is not prepared by deviding a genomic DNA into a control and a test DNA. In some embodiments, the control DNA is prepared by fabrication of a double-stranded synthetic template having the same DNA sequence as the corresponding genomic DNA except that at least some (e.g., at least one, most or all) cytosine residues are replaced by uracil or thymadine residues in pre-determined patterns reflecting possible unmethylated cytosines in the genomic DNA. In some embodiments, all non-CpG cytosine residues are replaced with uracil or thymidine residues.

### BRIEF DESCRIPTION OF FIGURES

FIG. 1 shows the potential states of methylation on the two DNA strands at one site in a diploid cell.

FIG. 2 is a schematic depicting the use of selective limiting primers with LATE-PCR and Lights-On/Lights-Off probes according to certain embodiments of the methods described herein.

FIG. 3 is a schematic depicting the use of a SuperSelective limiting primer with LATE-PCR and Lights-On/Lights-Off probes according to certain embodiments of the methods described herein.

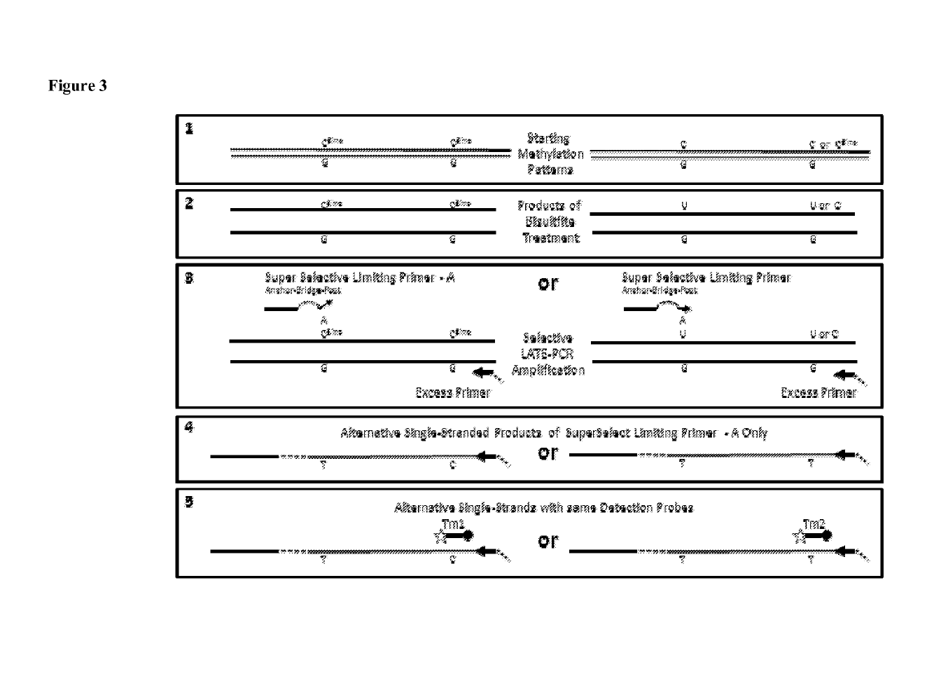

FIG. 4 is a schematic depicting the use of DISSECT with LATE-PCR and Lights-On/Lights-Off probes according to certain embodiments of the methods described herein.

FIG. 5 shows preferential amplification of 10,000 copies of mutant EGFR L858R DNA (Curve 1) relative to 10,000 copies of wild-type EGFR DNA (Curve 2).

FIG. 6 shows preferential amplification of 10,000 copies of mutant BRAF V600E DNA (Curve 3) relative to 10,000 copies of wild-type BRAF DNA (Curve 4).

FIG. 7 shows that increasing concentrations of EP003 did not appreciably affect amplification of EGFR L858R mutant targets (Curve 5, all EP003 concentrations) but preferentially delayed the amplification of wild-type EGFR targets (Curve 6, 0 nM EP003; Curve 7, 25 nM EP003; Curve 8, 50 nM EP003; Curve 9, 100 nM EP003).

FIG. 8 shows that increasing concentrations of EP003 did not appreciably affect amplification of BRAF V600E mutant targets (Curve 10, all EP003 concentrations) but preferentially delayed the amplification of wild-type BRAF targets (Curve 11, 0 nM EP003; Curve 12, 25 nM EP003; Curve 13, 50 nM EP003; Curve 14, 100 nM EP003).

FIG. 9 shows an exemplary temperature cycling profile used for SuperSelective primers in certain amplification reactions.

FIG. 10 is a schematic depicting the use of SuperSelective primers according to certain embodiments of the methods disclosed herein. When a SuperSelective primer is hybridized to an original target molecule, the bridge sequence in the primer remains single stranded and forms a bubble. Once a SuperSelective primers is successfully extended on the matched target sequence the resulting amplification product incorporates the entire SuperSelective primer sequence (including the bridge sequence). As a result, the melting temperature of the SuperSelective primer on the amplification product target is higher than its melting temperature on the original target sequence.

FIG. 11 is a schematic depicting the application of SuperSelective primers to LATE-PCR according to certain embodiments of the methods disclosed herein. SuperSelective primer and the reverse primers are converted to LATE-PCR primers and a 5′ tail non-complementary to the original target sequence is added to the LATE-PCR reverse primer such that, once incorporated into an amplification product, the melting temperature of the fully complementary reverse primer on the amplification product target is higher on than the melting temperature on the original target.

FIG. 12 shows an exemplary temperature cycling profile for SuperSelective primers in certain LATE-PCR amplification reactions.

FIG. 13 shows an exemplary temperature profile for SuperSelective primers in certain LEL-PCR amplification reactions. In this embodiment, a limiting SuperSelective primer undergoes several cycles of linear amplification with an annealing temperature of below the melting temperature of the SuperSelective primer on the original target sequence and above the melting temperature of the reverse primer on the original target sequence (e.g., 71° C.). A single cycle is then performed with an annealing temperature of below the melting temperature for the reverse primer on the original target sequence (e.g., 60° C.). This is followed by multiple cycles with an annealing temperature of above the melting temperatures of the SuperSelective primer and the reverse primer on the original target sequence, but below the melting temperatures of the SuperSelective primer and the reverse primer on the amplification product sequence (e.g., 75° C.) to enable amplification of amplification product targets first exponentially and then, once the limiting SuperSelective primer has been exhausted, linearly.

FIG. 14 shows preferential amplification of three replicates of 10,000 copies of matched unmethylated targets (Curves 15) relative to only one out of three replicates of 10,000 copies of mismatched methylated targets (Curves 16) after a single round of linear extension of the limiting SuperSelective primer at 70° C.

FIG. 15 shows that increasing the number of linear amplification cycles for the LATE-PCR SuperSelective limiting primer from one to ten allows better amplification of the matched (unmethylated) targets (Curves 17) but enough mismatched (methylated) targets hybridize under these conditions to allow amplification of all three replicates (Curves 18).

FIG. 16 shows that addition of 25 nM of the Reagent 2 increased the selectivity of the LATE-PCR SuperSelective primers by 0.8 Ct values. The delta Ct value between the matched unmethylated target (Curves 19) and the mismatched methylated target (Curves 20) was 7.3 cycles compared to the delta Ct value between the matched unmethylated target +25 nM Reagent 2 (Curves 21) and the mismatched methylated target+25 nM Reagent 2 (Curves 22).

FIG. 17 shows that Reagent 2 present in the samples from FIG. 16 can be readily visualized by virtue of its own fluorescence (Cal Orange, in this particular example). Curves 23 correspond to reactions without Reagent 2; Curves 24 corresponds to reactions with 25 nM Reagent 2.

FIG. 18 shows that the probe readily distinguished selectively amplified amplicons containing a simulated internal unmethylated site from those containing the same simulated site but methylated. Curves 25 correspond to amplicons with an internal unmethylated site, Curves 26 correspond to amplicons with an internal methylated site.

FIG. 19 shows that Hairpin Reagent 1 (Curve 27) prevented amplification over a range of temperature. Control reactions with Taq DNA polymerase+Taq DNA polymerase antibody demonstrate that failure to amplify was due to Hairpin Reagent 1 controlling the activity of Taq DNA polymerase at the annealing temperature (Curve 28).

FIG. 20 is a schematic depicting temperature imprecise (TI) PCR according to certain embodiments of the methods described herein.

### DETAILED DESCRIPTION

### General

Provided herein are methods using Linear After the Exponential Polymerase Chain Reaction (“LATE-PCR”) or Linear-Expo-Linear Polymerase Chain Reaction (“LEL-PCR”) to amplify specific DNA targets, and in particular to determine whether one or more CpG dinucleotides in a DNA molecule is methylated.

Contacting DNA with bisulfite under certain conditions results in the deamination of unmethylated cytosine residues, converting them to uracil in a process referred to herein as “bisulfite conversion”. Methylated cytosine (5-methylcytosine) is resistant to bisulfite conversion. Consequently, contacting a DNA molecule with a bisulfite solution under the proper conditions, described in greater detail herein below, will result in the majority of the unmethylated cytosine residues being converted to uracil while the majority of the methylated cytosine residues remain unchanged. Since essentially all cytosine residues that are not part of a CpG dinucleotide sequence are unmethylated, bisulfite conversion results in the majority of the cytosine residues in the DNA molecule being converted to uracil residues.

The fact that methylated cytosine residues are resistant to deamination during the bisulfite conversion process can be used to determine whether a cytosine residue in a specific CpG dinucleotide is methylated. In order to do this, the genomic DNA is subjected to bisulfite conversion and whether the cytosine residue was converted into a uracil residue is detected. To accomplish this, the region of the bisulfite-converted DNA sequence that contained the CpG dinucleotide of interest is amplified to produce an amplification product. During most amplification processes, the uracil residues formed during the bisulfite conversion process will be replaced by incorporation of thymidine residues during the course of amplification. Thus, whether a CpG was originally methylated can be determined by, for example, sequencing the amplification product or by hybridizing the amplification product with one or more probes that are able to distinguish between a cytosine residue and a thymidine residue at the original CpG position. Moreover, because DNA is comprised of two complementary antiparallel strands, the presence of a methylated or unmethylated cytosine in the original sample can also be judged by analysis of the complementary strand that will either have a guanosine residue or an adenine residue (complementary to cytosine and thymidine, respectively).

In certain embodiments, the methods described herein include three general steps: 1) contacting the DNA molecule with a bisulfite solution; 2) performing LATE-PCR or LEL-PCR amplification on the bisulfite-modified DNA molecule to produce a single-stranded DNA amplification product; and 3) determining whether the cytosine in the CpG dinucleotide was converted to uracil during step 1. In certain embodiments, step 3) comprises hybridizing one or more probes to the amplification product by lowering the temperature at the end of amplification without opening the vessel in which amplification takes place. In certain embodiments the target has a specific sequence which is distinct due to a polymorphism that does not arise from prior methylation.

In certain embodiments, the sample containing the genomic DNA to be analyzed is divided into two portions prior to analysis, with only one of the portions undergoing bisulfite conversion. In such embodiments both the bisulfite treated and untreated DNA samples are then amplified in parallel reactions by LATE-PCR or LEL-PCR and the resulting amplification products are analyzed by hybridization to one or more probes at end-point. The fluorescent signals generated using sets of probes are called fluorescent contours and these contours can be converted mathematically into fluorescent signatures. Fluorescent signatures vary in a base sequence dependent manner, even down to the level of single base changes. Comparison of the fluorescence signatures generated by the two amplification products can then be used to determine the methylation status of the CpG dinucleotide of interest. Comparison can include ratio analysis of different fluorescent signatures.

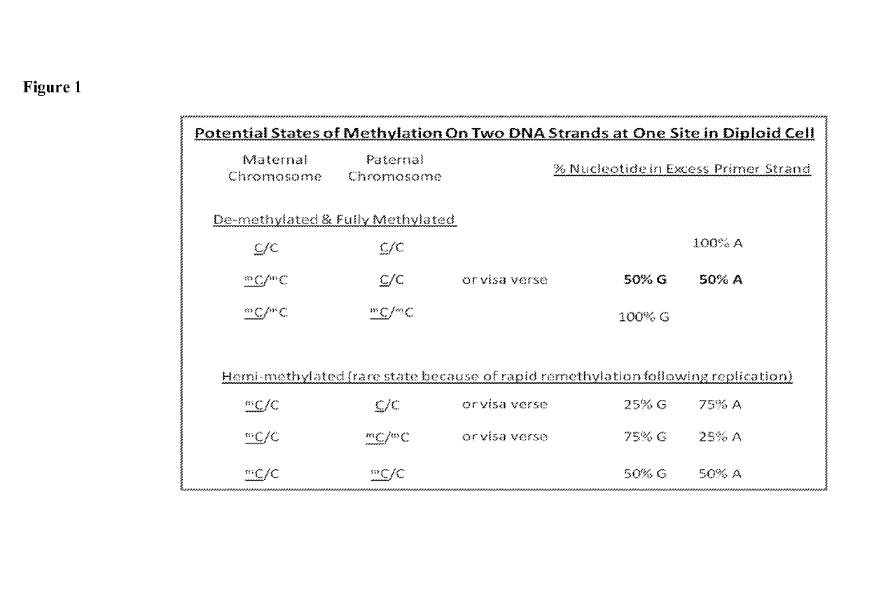

In some embodiments the methods described herein are able to distinguish between fully unmethylated, hemimethylated, and fully methylated CpG/GpC sequences at a particular site and/or at multiple sites within a single target sequence. The left side of FIG. 1 depicts the possible patterns of 5′methylcytosine at a single CpG site on each strand of double-stranded DNA in a diploid eukaryotic cell. Methylcytosine is not converted to uracil by bisulfite treatment, while cytosine is converted to uracil by bisulfite treatment. During amplification of bisulfite-converted DNA, unconverted methylcytosine nucleotides remain complemented by guanidine, but the newly formed uracil nucleotides are complemented by adenosine which, in turn are complemented by thymidine during the course of amplification. LATE-PCR or LEL-PCR generates an excess primer strand for each amplicon. Assuming that the nucleotides underlined on the left side of FIG. 1 are the template for subsequent excess primer strand synthesis, it is possible to predict the number of expected G and A residues in the excess primer strand, provided that all possible patterns of methylation/demethylation are equally likely. In some embodiments the DNA sequences of both strands are analyzed.

In some embodiments provided herein is a single-tube method of LATE-PCR or LEL-PCR amplification, detection, and analysis of at least one protein-free genomic DNA target sequence known to contain two or more CpG dinucleotides modulated by methylation, or the bisulfite converted version of this DNA target. In some embodiments, the method includes the step of performing PCR using, for example, a LATE-PCR or LEL-PCR reaction mixture containing the reagents required for amplification, including at least one Excess Primer (e.g., a tailed Excess pPrimer with 5′ sequence that is not complementary to the target sequence), and at least one Limiting Primer (e.g., a SuperSelective primer), to generate amplicons of the DNA target in a closed-tube homogeneous reaction that also contains at least one probe capable of quantitative end-point temperature-dependent preferential hybridization to a complementary sequence containing the dinucleotide CpG or its alternative a TpG dinucleotide, or, the dinucleotide GpC or its alternative a ApC dinucleotide. In some embodiments, the method includes the step of lowering the temperature of the closed tube to one or more temperatures at least 5° C. below the melting temperature of the Limiting primer to its target to achieve hybridization of the probes to the amplicon. In some embodiments, the method includes the step of measuring the fluorescence emitted from the resulting probe/target hybrids at a plurality of temperatures, at intervals of 5° C. or less, to establish whether each of the probes has hybridized to a CpG dinucleotide CpG or its alternative a TpG dinucleotide, or a GpC dinucleotide or its alternative a ApC dinucleotide.

In some embodiments provided herein is a single-tube method of LATE-PCR or LEL-PCR preferential amplification, detection, and analysis, of at least one protein-free genomic DNA target sequences known to contain two or more or three or more CpG dinucleotides modulated by methylation, or the bisulfite converted version of this DNA target. In some embodiments, the method includes the step of performing a LATE-PCR or LEL-PCR reaction, using, for example, a LATE-PCR reaction mixture or a LEL-PCR reaction mixture containing the reagents required for amplification, including at least one Excess Primer, and at least one Limiting Primer, wherein the Limiting Primer exhibits sequence specificity in that it preferentially initiates amplification of the target sequence due to the presence of a CpG dinucleotide or its alternative, a UpG dinucleotide, to generate amplicons of the DNA target due to the sequence specificity of the Limiting primer in a closed-tube homogeneous reaction that also contains at least one probe capable of quantitative end-point temperature-dependent preferential hybridization to a complementary sequence containing the dinucleotide CpG or its alternative a TpG dinucleotide, or, the dinucleotide GpC or its alternative a ApC dinucleotide. In some embodiments, the method includes the step of lowering the temperature of the closed tube to one or more temperatures at least 5° C. below the melting temperature of the Limiting primer to its target to achieve hybridization of the probes to the amplicon. In certain embodiments the method includes the step of measuring the fluorescence emitted from the resulting probe/target hybrids at a plurality of temperatures, at intervals of 5° C. or less, to establish whether each of the probes has hybridized to a CpG dinucleotide CpG or its alternative a TpG dinucleotide, or a GpC dinucleotide or its alternative a ApC dinucleotide.

In certain embodiments, provided herein is a single-tube method of LATE-PCR or LEL-PCR preferential amplification, detection, and analysis, of at least one protein-free genomic DNA target sequences known to contain two or more or three or more CpG dinucleotides modulated by methylation, or the bisulfite converted version of this DNA target. In some embodiments the method includes the step of performing a LATE-PCR or LEL-PCR amplification using, for example, a reaction mixture containing the reagents required for amplification, including at least two primers and at least one reagent capable of increasing polymerase selectivity throughout the reaction, to generate amplicons of at least one strand of the DNA target in an initial closed-tube homogeneous amplification reaction. In some embodiments the method includes the step of accessing the tube (e.g., opening the lid of the tube) and adding magnetic beads having a covalently linked capture probe capable of temperature-dependent preferential hybridization to a complementary sequence containing the dinucleotide CpG or its alternative a TpG dinucleotide, or, the dinucleotide GpC or its alternative a ApC dinucleotide in the amplicon. In certain embodiments the method includes the step of adding additional magnetic beads with capture probes to the complementary strands. In some embodiments, the method includes the step of altering the temperature to selectively release into the supernatant only those products strands that have a mis-match to their capture probe. In some embodiments, the method includes the step of combining the released strand with at least one Excess Primer and at least one Selective Limiting Primer that hybridize to the amplicon, and at least two probes capable of quantitative end-point temperature-dependent preferential hybridization to a complementary sequence in the amplicon containing the dinucleotide CpG or its alternative a TpG dinucleotide, or the dinucleotides GpC or its alternative a ApC dinucleotide. In some embodiments, the method includes the step of sealing the tube and running LATE-PCR or LEL-PCR amplification to generate additional copies of the amplicon. In some embodiments, the method includes the step of lowering the temperature of the closed tube to one or more temperatures at least 5° C. below the melting temperature of the Limiting primer to achieve hybridization of the probes to the amplicons. In some embodiments, the method includes the step of measuring the fluorescence emitted from the resulting probe/target hybrids at a plurality of temperatures, at intervals of 5° C. or less, to establish whether the dinucleotide CpG or its alternative TpG is present at each site, or, GpC or its alternative ApC is present at each site.

In some embodiments provided herein are kits for performing one or more of the methods described herein. In some embodiments the kits contain one or more reagents described herein and instructions for performing the method described herein.

### DEFINITIONS

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the terms “hybridize” or “hybridization” refer to the hydrogen bonding of complementary DNA and/or RNA sequences to form a duplex molecule. As used herein, hybridization generally takes place under conditions that can be adjusted to a level of stringency that reduces or even prevents base-pairing between a first oligonucleotide primer or oligonucleotide probe and a target sequence, if the complementary sequences are mismatched by as little as one base-pair. In a closed tube reaction, the level of stringency can be adjusted by changing temperature and, as a result, the hybridization of a primer or a probe to a target can occur or not occur depending on temperature. Thus, for example, a probe or a primer that is mismatched to a target can be caused to hybridize to the target by sufficiently lowering the temperature of the solution.

As used herein, the Tm or melting temperature of two oligonucleotides is the temperature at which 50% of the oligonucleotide/targets are bound and 50% of the oligonucleotide target molecules are not bound. Tm values are concentration dependent and are affected by the concentration of monovalent, divalent cations in a reaction mixture. Tm can be determined empirically or calculated using the nearest neighbor formula, as described in Santa Lucia, J. PNAS (USA) 95:1460-1465 (1998), which is hereby incorporated by reference. LATE-PCR takes into account the concentration-adjusted melting temperature of the Limiting Primer at the start of amplification, Tm_{[0]}^{L}, the concentration-adjusted melting temperature of the Excess Primer at the start of amplification, Tm_{[0]}^{X}, and the concentration-adjusted melting temperature of the single-stranded amplification product, Tm_{A}. For LATE-PCR primers, Tm_{[0]} can be determined empirically, as necessary when non-natural nucleotides are used, or calculated according to the nearest neighbor method using a salt concentration adjustment. For LATE-PCR the melting temperature of the amplicon is calculated using the formula: Tm=81.5+0.41 (% G+% C)−500/L+16.6 log [M]/(1+0.7[M]), where L is the length in nucleotides and [M] is the molar concentration of monovalent cations.

As used herein, the term “bisulfite conversion” refers to the process of contacting DNA with bisulfite under conditions such that the majority of the unmethylated cytosine residues in the DNA are converted to uracil but the majority of the methylated cytosine residues (5-methylcytosine) are not converted to uracil.

As used herein, the term “Linear-After-The Exponential PCR” or “LATE-PCR” refers to a non-symmetric PCR method that utilizes unequal concentrations of primers and yields single-stranded primer-extension products (referred to herein as amplification products or amplicons). LATE-PCR is described, for example, in U.S. Pat. Nos. 7,198,897 and 8,367,325, each of which is incorporated by reference in its entirety.

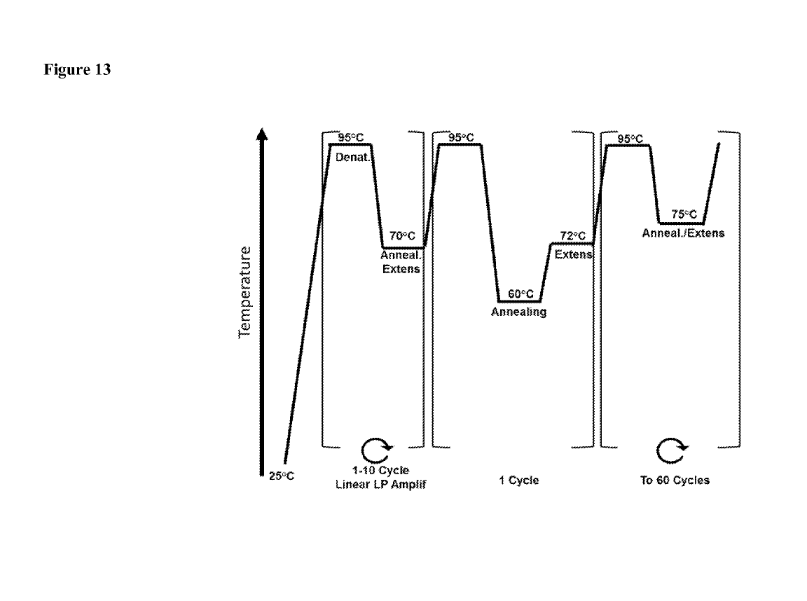

As used herein, the term “Linear-Expo-Linear PCR” or “LEL-PCR” refers to a PCR method in which a target nucleic acid sequence undergoes an initial linear amplification process producing an amplification product that is then selectively subjected to LATE-PCR. LEL-PCR is performed using a limiting forward primer and an excess reverse primer that each have incomplete sequence complementarity or sequence identity to a region of the target nucleic acid sequence. During LEL-PCR, the target nucleic acid sequence is first linearly amplified under conditions at which the forward primer but not the reverse primer anneals to and amplifies the target nucleic sequence. Following linear amplification, at least one low annealing temperature cycles are performed under conditions at which both the forward primer and the reverse primer anneal to and amplify the target nucleic acid sequence or its complement. Following the low annealing temperature cycle(s), a series of LATE-PCR cycles are performed under conditions at which both the forward primer and the reverse primer anneal to the amplification products produced during the low annealing temperature cycle(s), but not the original target nucleic acid sequence or its complement. An exemplary LEL-PCR process is depicted in FIG. 13.

As used herein, Low-Tm probes and Superlow-Tm probes are fluorescently tagged, electrically tagged or quencher tagged oligonucleotides that have a Tm of at least 5° C. below the mean primer annealing temperature during exponential amplification of a LATE-PCR amplification. In some embodiments sets of signaling and quencher Low-Tm and Superlow-Tm probes are included in LATE-PCR amplification mixtures prior to the start of amplification. There are many possible designs of Low-Tm and Superlow-Tm probes. Molecular beacons, for example, can be designed to be Low-Tm probes by designing them with shorter stems and loops compared standard molecular beacons that hybridize to target strands at or above the primer annealing temperature of the reaction.

As used herein, the term “Lights-On/Lights-Off probes” refers to a probe set that hybridize to adjacent nucleic acid sequences on the single-stranded DNA target to be detected Lights-On/Lights-Off probe technology is more fully described in PCT application No. PCT/US10/53569, hereby incorporated by reference in its entirety.

As used herein, Lights-Off Only probes are probes labeled with a non-fluorescent quencher moiety (e.g., a Black Hole quencher) that hybridize to a single-stranded DNA target to be detected. Lights-Off Only probes are used in combination with a fluorescent dye that binds preferentially to double-stranded DNA (e.g., SYBR® Green dye) to detect single-stranded amplification products (e.g., single-stranded DNA products produced by LATE-PCR). This is done by subjecting an amplified sample containing the fluorescent ds-DNA dye and the Lights-Off Only probe at multiple temperatures that are below the melting temperature of the probe to excitation at a wavelength appropriate for stimulating the dye and detecting emission at a wavelength appropriate for detecting emission from the dsDNA-dye. Lights-Off Only probe technology is more fully described in U.S. Provisional Application No. 61/702,019, hereby incorporated by reference in its entirety.

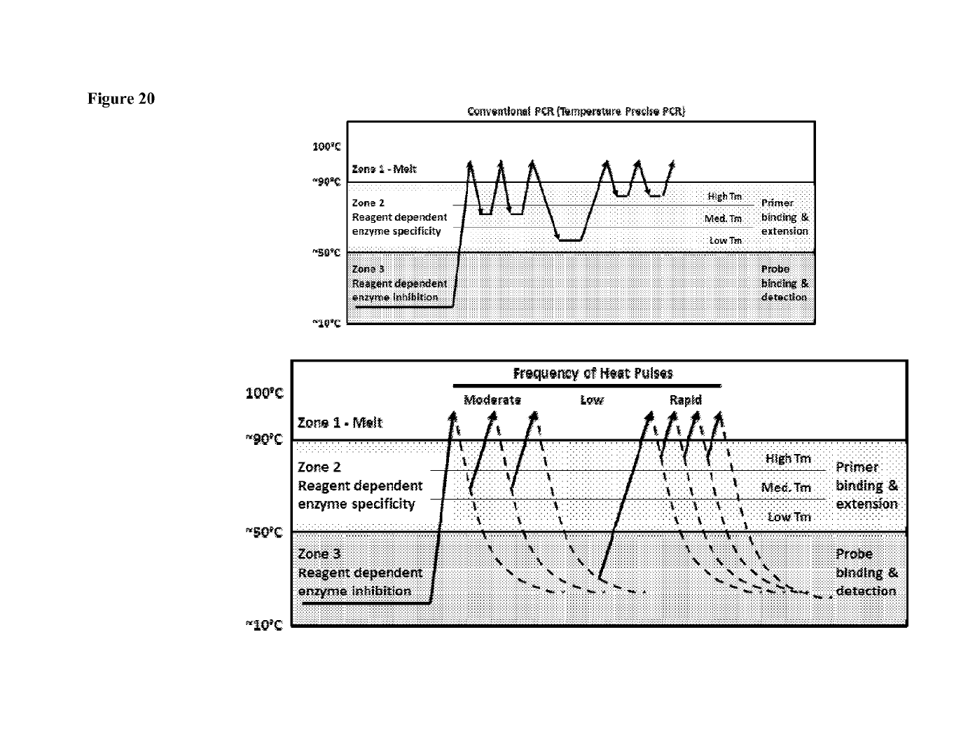

As used herein, the term “Temperature Imprecise PCR” or “TI-PCR” refers to a PCR amplification method in which the temperature of the reaction vessel is elevated by heating at time-adjustable intervals for time-adjustable lengths of time and in which the temperature of the reaction vessel is decreased via passive cooling for time-adjustable intervals. The principles of TI-PCR can be applied to other PCR amplification techniques, including LATE-PCR and LEL-PCR. An exemplary TI-PCR process is depicted in FIG. 20.

### Bisulfite Conversion

As described above, “bisulfite conversion” refers to the process of contacting DNA with bisulfite under conditions such that the majority of the unmethylated cytosine residues in the DNA are converted to uracil but the majority of the methylated cytosine residues (5-methylcytosine) are not converted to uracil. In certain embodiments the conditions are such that greater than 50%, 60%, 70%, 75%, 80%, 85%, 90%, 95%, 96%, 97%, 98% or 99% of unmethylated cytosine residues are converted to uracil. In some embodiments less than 50%, 40%, 30%, 25%, 20%, 15%, 10%, 5%, 4%, 3%, 2% or 1% of methylated cytosine residues are converted to uracil. In general, only certain cytosine residues that are a part of a CpG dinucleotide can be methylated and essentially all other cytosine residues are unmethylated. As a result of the sequence changes that result from the bisulfite conversion process, the two strands of the original DNA molecule are no longer able to perfectly base-pair.

In certain embodiments, bisulfite conversion is performed on substantially protein free genomic DNA. Any appropriate method known in the art can be used to prepare the substantially protein free genomic DNA. Examples of standard laboratory protocols for preparing a protein-free DNA solution are provided, for example, in Sambrook et al., Molecular Cloning: A Laboratory Manual, 2nd edition, Cold Spring Harbor Laboratory Press (1989), incorporated by reference in its entirety. Additionally, numerous commercially available kits are available for use in isolating genomic DNA, including genomic isolation kits manufactured by Promega®, Invitrogen® and Qiagen®. In some embodiments substantially protein free genomic DNA is prepared using QuantiLyse, a proteinase K lysis solution optimized for PCR containing 10 mM Tris-Cl pH 8.3, 5 μM SDS, and 0.1 mg/ml Proteinase K (See, e.g., Pierce et al., Biotechniques 32:1106-1111 (2002), incorporated by reference in its entirety). In some embodiments protein free DNA is prepared by separation of the DNA and the protein through the use of a high concentration of a chaotropic salt as described in U.S. Pat. No. 7,465,562, which is hereby incorporated by reference in its entirety. In some embodiments protein free DNA is prepared in the same tube or vessel in which a PCR and/or LATE-PCR amplification is performed following a dilution of the sample. In some embodiments, the protein-free DNA comprises between 1-10,000 genomes of DNA.

In certain embodiments the DNA to be analyzed is broken into one or more smaller pieces (“fragmented”) prior to bisulfite conversion. In certain embodiments, between 250 ng and 2 μg of the genomic DNA is fragmented. In some embodiments the genomic DNA is cut with a restriction enzyme that cleaves the genomic DNA outside of the region to be amplified by LATE-PCR. In certain embodiments the fragment of genomic DNA containing the region to be amplified is cut such that it is less than 1,000, 900, 800, 700, 600, 500, 450, 400, 350, 300, 250, 200, 150, 100, 75 or 50 bp in length. In some embodiments the genomic DNA is fragmented into smaller pieces using an essentially random process, such as sonication. In certain embodiments the sonication conditions are such that it reduces the average size of the genomic DNA fragments to less than 1,000, 900, 800, 700, 600, 500, 450, 400, 350 or 300 bp in length. In certain embodiments, the average size of the fragments remains at least 200, 150, 100, 75 or 50 bp in length following fragmentation. In some embodiments the fragmented DNA is formed as a result of formalin fixation. In some embodiments the DNA is fragmented through a natural process, such as apoptosis. The resulting DNA fragments can be observed in plasma and/or serum.

In some of the embodiments at least one portion of genomic DNA (“the bisulfite converted DNA solution”) undergoes bisulfite conversion, while at least one other solution (“the control DNA solution”) does not undergo bisulfite conversion. The control DNA solution can be derived from the genomic DNA or from synthetic DNA. The LATE-PCR amplification and Lights-On/Lights-Off probe hybridization protocols described herein may be performed on both the bisulfite converted DNA solution and the control DNA solution.

In some embodiments any method known in the art can be used in the methods described herein for the bisulfite conversion of the genomic DNA. Exemplary bisulfite conversion protocols are available, for example, in Hajkova et al., Methods Mol. Biol. 200:143-154 (2002) and Mill et al., Am. J. Hum. Genet., 82:696-711 (2008). A bisulfite conversion protocol is also available from Dr. Axel Shumacher at: http://www.methylogix.com/genetics/protocols.shtml-Dateien/schumachersguide1.html. Kits for performing bisulfite conversion are also available, including the EpiMark® bisulfite conversion kit from New England Biolabs, the Cells-to-CpG™ bisulfite conversion kit from Life Technologies, the EpiTect™ bisulfite conversion kit from Qiagen and the CpGenome™ bisulfite conversion kit from Millipore.

### LATE-PCR

In certain embodiments LATE-PCR is used to amplify a region of bisulfite modified DNA (e.g., DNA in the bisulfite modified DNA solution) and/or control DNA (e.g., DNA in the control DNA solution). LATE-PCR is a non-symmetric PCR method that utilizes unequal concentrations of primers and yields single-stranded primer-extension products (amplification products). LATE-PCR utilizes the basic steps of strand melting, primer annealing, and primer extension by a DNA polymerase caused or enabled to occur repeatedly by a series of temperature cycles. However, in the early cycles of a LATE-PCR amplification, when both primers are present, LATE-PCR amplification amplifies both strands of a target sequence exponentially, as occurs in conventional PCR, but in the later cycles, when only one primer is present, only one strand of the target sequence amplified linearly.

In certain embodiments, sets of LATE-PCR primers specific for the regions to be tested are designed according to LATE-PCR design criteria as set forth in Sanchez et al., Proc Natl Acad Sci USA 101:1933-1938 (2004) and/or Pierce et al., Proc Natl Acad Sci USA 102:8609-8614 (2005), each of which is hereby incorporated by reference in its entirety. When multiple amplifications are being performed (for example, when two different regions are being analyzed simultaneously), primers can be designed to accommodate amplification under the same reaction conditions with similar priming efficiencies. In some embodiments the primers are located in sequences neighboring the one or more CpG dinucleotides to be analyzed (for example, outside a CpG-rich island). In certain embodiments the primers length is less than 200, 175, 150, 125, 100, 90, 80, 70, 60 or 50 nucleotides in length to facilitate amplification of DNA, which may have been fragmented either prior to or during bisulfite conversion. Primers used to amplify control DNA have sequence complementary to the native DNA sequence, while primers used to amplify bisulfite treated DNA have sequence complementarity bisulfite modified DNA (e.g., nucleotides complementary to cytosine residues in the native genomic DNA sequence will instead be complementary to thymidine residues).

In some embodiments in which LATE-PCR amplification is performed on both control DNA and bisulfite-modified DNA, primers are designed such that the double-stranded amplification products produced during the exponential phase of amplification of the control DNA has a similar melting temperature to the double-stranded amplification product produced during exponential phase of amplification of the bisulfite converted DNA. In some embodiments the melting temperature of the control DNA amplification product is within less than 5, 4, 3, 2, 1, 0.5, 0.4, 0.3, 0.2 or 0.1 degrees of the bisulfite converted DNA amplification product.

### LEL-PCR

In certain embodiments LEL-PCR is used to amplify a region of bisulfite modified DNA (e.g., DNA in the bisulfite modified DNA solution) and/or control DNA (e.g., DNA in the control DNA solution).

LEL-PCR is a PCR method in which a sample containing a target nucleic acid is subjected to amplification conditions such that the target nucleic acid sequence first undergoes one or more rounds (e.g., 1-10 rounds) of a linear amplification process to produce a single-stranded amplification product containing a sequence complementary to the target nucleic acid sequence. The sample is then subjected to conditions such that the single-stranded amplification product is subjected to one or more rounds (in some embodiments one round) of reverse strand synthesis, followed by one or more rounds of an exponential amplification process at an elevated temperature to produce a double-stranded amplification product in which a first strand contains a sequence complementary to the target nucleic acid sequence and a second strand contains a sequence corresponding to the target nucleic acid sequence and complementary to the sequence of the first amplification product strand. Following exponential amplification, the double-stranded amplification product is then subject to a linear amplification process in which a second single-stranded amplification product is generated. In certain embodiments, the second single-stranded amplification product will contain a sequence corresponding to the target sequence.

In certain embodiments, LEL-PCR is performed by combining a first primer, referred to as the forward primer and a second primer, referred to as a reverse primer, with a target nucleic acid and amplification reagents. The first and second primers are designed such that they are not perfectly complementary to the target nucleic acid sequence. For example, in some embodiments, the first and/or second primer have an internal non-complementary region (e.g., SuperSelective primers), have a non-complementary 5′ region, and/or contain sequence mismatches with respect to the target nucleic acid sequence. In some embodiments, the forward primer is a SuperSelective primer and the reverse primer has a non-complementary 5′ region. The first and second primers therefore have a first melting temperature on the imperfectly complementary target sequence and a second, higher melting temperature on a perfectly complementary sequence. In certain embodiments, the melting temperature of the forward primer on the target nucleic acid sequence is higher than the melting temperature of the reverse primer on the target nucleic acid sequence. In some embodiments, the melting temperature of the reverse primer on a perfectly complementary sequence is higher than the melting temperature of the forward primer on the target nucleic acid sequence. In some embodiments, the forward primer is a limiting primer and is included in the reaction solution at a lower molar concentration than the reverse primer. An exemplary LEL-PCR process is depicted in FIG. 13.

In some embodiments, the LEL-PCR process includes an initial linear amplification phase. In certain embodiments, the initial linear amplification phase includes one or more linear amplification cycles. In some embodiments, the initial linear amplification phase includes at least 1, 2, 3, 4, 5, 6, 7, 8, 9 or 10 cycles. In some embodiments, the initial linear amplification phase includes between 1 and 10 cycles. In some embodiments, each cycle of the linear amplification phase includes at least a denaturation step and an annealing step. In some embodiments, each cycle also includes an extension step. In some embodiments, the annealing step and the extension step are combined into a single step. In some embodiments, the denaturation step is performed at a temperature above the melting temperature of the forward primer on the target nucleic acid sequence. In some embodiments, the denaturation step is performed at a temperature above the melting temperature of the forward primer for a perfectly complementary sequence. In some embodiments, the denaturation step is performed at between 90° C. and 100° C. In some embodiments, the denaturation step is performed at a temperature of about 95° C. In some embodiments, the annealing step is performed at a temperature that is below the melting temperature of the forward primer on the target nucleic acid sequence but above the melting temperature of the reverse primer on the target nucleic acid sequence. In some embodiments, the annealing step is combined with an extension step, and the extension step is performed at a temperature at which a polymerase enzyme in the reaction solution is active (e.g., between about 70° C. and 75° C.). In some embodiments, the annealing step is followed by an extension step that is performed at a temperature at which a polymerase enzyme in the reaction solution is active (e.g., between about 70° C. and 75° C.).

In some embodiments, the initial linear amplification phase is followed by one or more low annealing temperature cycles. In some embodiments, the initial linear amplification phase is followed by a single low annealing temperature cycle. In some embodiments, the low annealing temperature cycle includes at least a denaturation step and an annealing step. In some embodiments, the cycle also includes an extension step. In some embodiments, the annealing step and the extension step are combined into a single step. In some embodiments, the denaturation step is performed at a temperature above the melting temperature of the forward primer on the target nucleic acid sequence. In some embodiments, the denaturation step is performed at a temperature above the melting temperature of the forward primer for a perfectly complementary sequence. In some embodiments, the denaturation step is performed at between 90° C. and 100° C. In some embodiments, the denaturation step is performed at a temperature of about 95° C. In some embodiments, the annealing step is performed at a temperature that is below the melting temperature of the reverse primer on the target nucleic acid sequence. In some embodiments, the annealing step is combined with an extension step, and the extension step is performed at a temperature at which a polymerase enzyme in the reaction solution is active (e.g., between about 70° C. and 75° C.). In some embodiments, the annealing step is followed by an extension step that is performed at a temperature at which a polymerase enzyme in the reaction solution is active (e.g., between about 70° C. and 75° C.).

In some embodiments, the low annealing temperature cycle is followed by performance of a LATE-PCR phase that includes an exponential amplification phase and a linear amplification phase. In certain embodiments, the LATE-PCR phase includes one or more amplification cycles. In some embodiments, LATE-PCR phase includes at least 10, 15, 20, 25, 30, 35, 40, 45, 50, 55 or 60 cycles. In some embodiments, the early cycles of the LATE-PCR phase, when both the forward primer and the reverse primer are present, both strands of a target sequence are amplified exponentially, as occurs in conventional PCR, but in the later cycles, when only one primer is present, only one strand of the target sequence amplified linearly. In some embodiments, the forward primer is limiting. In some embodiments, each cycle of the LATE-PCR phase includes at least a denaturation step and an annealing step. In some embodiments, each cycle also includes an extension step. In some embodiments, the annealing step and the extension step are combined into a single step. In some embodiments, the denaturation step is performed at a temperature above the melting temperature of both the forward primer and the reverse primer on perfectly complementary nucleic acid sequences. In some embodiments, the denaturation step is performed at between 90° C. and 100° C. In some embodiments, the denaturation step is performed at a temperature of about 95° C. In some embodiments, the annealing step is performed at a temperature that is above the melting temperature of the forward primer on the target nucleic acid sequence but below the melting temperature of the forward primer on a perfectly complementary nucleic acid sequence. In some embodiments, the annealing step is performed at a temperature that is above the melting temperature of the reverse primer on the target nucleic acid sequence but below the melting temperature of the reverse primer on a perfectly complementary nucleic acid sequence. In some embodiments, the annealing step is combined with an extension step, and the extension step is performed at a temperature at which a polymerase enzyme in the reaction solution is active (e.g., between about 70° C. and 75° C.). In some embodiments, the annealing step is followed by an extension step that is performed at a temperature at which a polymerase enzyme in the reaction solution is active (e.g., between about 70° C. and 75° C.).

When multiple amplifications are being performed (for example, when two different regions are being analyzed simultaneously), LEL-PCR primers can be designed to accommodate amplification under the same reaction conditions with similar priming efficiencies. In some embodiments the primers are located in sequences neighboring the one or more CpG dinucleotides to be analyzed (for example, outside a CpG-rich island). In certain embodiments the primers length is less than 200, 175, 150, 125, 100, 90, 80, 70, 60 or 50 nucleotides in length to facilitate amplification of DNA, which may have been fragmented either prior to or during bisulfite conversion. Primers used to amplify control DNA have sequence complementary to the native DNA sequence, while primers used to amplify bisulfite treated DNA have sequence complementarity bisulfite modified DNA (e.g., nucleotides complementary to cytosine residues in the native genomic DNA sequence will instead be complementary to thymidine residues).

In some embodiments in which LEL-PCR amplification is performed on both control DNA and bisulfite-modified DNA, primers are designed such that the double-stranded amplification products produced during the exponential phase of amplification of the control DNA has a similar melting temperature to the double-stranded amplification product produced during exponential phase of amplification of the bisulfite converted DNA. In some embodiments the melting temperature of the control DNA amplification product is within less than 5, 4, 3, 2, 1, 0.5, 0.4, 0.3, 0.2 or 0.1 degrees of the bisulfite converted DNA amplification product.

### TI-PCR

In certain embodiments TI-PCR is used to amplify a region of bisulfite modified DNA (e.g., DNA in the bisulfite modified DNA solution) and/or control DNA (e.g., DNA in the control DNA solution). TI-PCR is a PCR amplification method in which the temperature of the reaction vessel is elevated by heating at time-adjustable intervals for time-adjustable lengths of time and in which the temperature of the reaction vessel is decreased via passive cooling for time-adjustable intervals. The principles of TI-PCR can be applied to other PCR amplification techniques, including LATE-PCR and LEL-PCR.

Conventional PCR processes, in which all temperatures in a thermal cycle are carefully controlled, can be regarded as Temperature Precise PCR, regardless of whether the format of the reaction is symmetric, asymmetric, or LATE-PCR. Most devices designed for performing conventional PCR processes (e.g., thermocyclers) use electrical energy to precisely control heating and cooling in order to achieve the high and the low temperatures required at each step in a thermal cycle, as well as the length of time at each temperature (e.g., as depicted in FIG. 20).

As disclosed herein, in certain embodiments, TI-PCR significantly reduces the need to precisely control both the temperature used to melt nucleic acids strands and the lower temperature used to control the hybridization of strands, (e.g., as depicted in FIG. 20). In certain embodiments, TI-PCR includes increasing the temperature of the reaction vessel increased by heating the vessel at time-adjustable intervals and for time-adjustable lengths of time. The temperature of the reaction vessel is decreased via passive cooling for time-adjustable intervals. In some embodiments, active cooling is not used in at least half of the TI-PCR cycles. In some embodiments, active cooling is not used in any of the TI-PCR cycles. The length of the interval between the heat pulses determines the temperature to which the reaction falls during the passive cooling. In some embodiments, enzyme activity (e.g., its activity or inactivity at various temperatures) is regulated using temperature-dependent reagents that interact with the enzyme in a temperature dependent manner. In some embodiments, primers are used that have a relatively low melting temperature on the target nucleic acid sequence and a higher melting temperature on a perfectly complementary DNA sequence (e.g., a SuperSelective primer). These primers therefore hybridize to and extend on template strands at different temperatures at different points in the reaction. Examples of these properties of TI-PCR are illustrated in FIG. 20.

In some embodiments, TI-PCR can be performed using symmetric PCR, asymmetric PCR, or non-symmetric PCR (LATE-PCR) depending on initial concentrations and melting temperatures of the primers used. In some embodiments, TI-PCR can be performed using LEL-PCR.

In some embodiments, TI-PCR differs from conventional PCR in one or more of the following ways: 1) thermal cycling takes place in a device that only has the capacity to heat the sample; and 2) cooling at each step depends on the passive loss of heat. In some embodiments, the high temperature applied to the sample is not precisely controlled by the device other than by the length of time for which heat is applied. In some embodiments, the low temperature applied to the sample is not precisely controlled by the device other than by the length of time between heating cycles. In some embodiments, enzyme activity/specificity/and inactivity is determined by the presence of temperature-dependent reagents that interact with the enzyme. Such reagents are described, for example, in the following patents and patent applications: U.S. Pat. No. 7,517,977; U.S. patent application publication No. 2012/0088275; and U.S. provisional patent application No. 61/755,872, each of which are herein incorporated by reference in its entirety.

In some embodiments, because TI-PCR only depends on active heating, it is ideally suited for use in resource poor settings that have limited electric power. Moreover, in some embodiments, TI-PCR can be used under conditions in which the ambient temperature varies from run to run, or even during a run.

In some embodiments, devices that run TI-PCR reactions can be very simple in design. For example a reaction tube can be attached to a mechanism that rotates the tube through a hot water bath and then through the air at varying rates, or a sample can be applied to a heating element that cycles between an on state and an off state at varying rates. In some embodiments, the passive dissipation of heat away from the reaction vessel is enhanced by making the volume of the sample small and/or by making the walls of the reaction chamber thin.

### SuperSelective Primers

In certain embodiments, SuperSelective primers are used in the methods described herein. Each SuperSelective primer has an anchor sequence, a bridge sequence, and a foot sequence that terminates in an extendable 3′ end. The anchor sequence can be of variable length, depending on the desired melting temperature to its target sequence. In some embodiments, the anchor is perfectly complementary to its designated initial target sequence. The bridge sequence is not complementary to the designated initial target sequence and, in some embodiments, has the fewest possible intra-molecular hybridization hairpins. In certain embodiments, the bridge sequence is between 14 and 45 nucleotides in length. In some embodiments, the bridge sequence can be adjusted as needed. For example, if several SuperSelective primers are used in the same reaction their bridge sequences are generally not the same. In some embodiments, the foot sequence is short, generally 5-8 nucleotides long. The foot sequence is perfectly complementary to a sequence within the designated initial target sequence that is some distance downstream, i.e. 3′, to the sequence that is complementary to the anchor of the same SuperSelective primer. In some embodiments, the foot is mis-matched to all allelic variants of the target sequence. Thus, a SuperSelective primer initially hybridizes to its designated target sequence by both the anchor sequences and the foot sequence. Under the same experimental conditions hybridization of the same SuperSelective primer to all allelic variants of the target sequence is less stable, because the foot portion of the primer is not fully complementary. Extension of the 3′end of a SuperSelective primer on its perfectly complementary target sequence is therefore more likely than extension on any allelic variant of the same target.

In some embodiments, a SuperSelective primer can be regarded to be the forward primer in a symmetric PCR amplification. In some embodiments, a second primer is used as the reverse primer in such a reaction. The target of the reverse primer lies downstream (3′) within the strand generated by extension of the SuperSelective primer, the Super-Select-Primer-Strand. During PCR amplification hybridization of the reverse primer to the Super-Select-Primer-Strand is followed by extension of the 3′ end of the reverse primer back to and through the entire length of the SuperSelective primer, thereby generating a Reverse Primer Strand that includes the complement of the foot, the bridge, and the anchor of the SuperSelective Primer. The 5′ end of the Reverse Primer is fully complementary to the Super-Selective-Primer-Strand. In some embodiments, the Reverse Primer and SuperSelective Primer are added to the Symmetric PCR reaction mixture at the same initial concentration, and used at a constant annealing temperature at which both primers hybridize to their respective target sequences.

In some embodiments, SuperSelective primers are used in LATE-PCR amplification. LATE-PCR utilizes a Limiting Primer and an Excess Primer which differ in their initial concentrations by at least 2-fold, 3-fold, 4-fold, 5-fold, 6-fold, 7-fold, 8-fold, 9-fold or 10-fold (e.g., at least 5-fold). In some embodiments, the initial concentration dependent melting temperatures of the Limiting primer and the Excess primer adhere to the following equation Tm^{L}−Tm^{X}≧0. In some embodiments, the relationship between the melting temperature of the amplicon Tm^{A} and the melting temperature of the Excess primer is described by the equation Tm^{A}−Tm^{X}≦25° C.

In some embodiments, the SuperSelective Primer serves as the Limiting primer and has an initial concentration of about 50 nM. In some embodiments, the first round of amplification the Tm of the anchor to the designated target sequence is about 71° C. and in all subsequent rounds of replication the Tm of the SuperSelective Primer to its full length complementary sequence is about 91° C.

In some embodiments, a SuperSelective primer is used in a LEL-PCR reaction. In some embodiments, the reverse primer serves as the excess primer. In some embodiments, the excess primer has extended non-complementary 5′ sequence that only hybridizes to the subsequent product of SuperSelective primer extension. Thus, in some embodiments, the initial Tm of the Excess Primer at an initial concentration of 1000 nM is 60° C., while in subsequent rounds of replication the Tm of the Excess primer to its full length complementary sequence is 81° C. In some embodiments, the initial melting temperatures of the amplicon (87° C.) and that of the Excess Primer (60° C.) fall outside of the bounds of LATE-PCR.

In some embodiments, the combined properties of the SuperSelective primer and the reverse primer allow for the introduction of novel temperature steps into the amplification protocol that facilitate greater stringency of hybridization between a primer and a designated target sequence and compared to its allelic variants. For example, in some embodiments, the one or more cycles of amplification can use an annealing temperature that is above the annealing temperature of the reverse primer for the target nucleic acid sequence (e.g., 70° C.), which therefore results in one or more rounds of linear amplification of the Super-Selective-Primer-Strand only. In some embodiments, the annealing temperature can then be lowered for one or more cycles to a temperature below the melting temperature of the reverse primer on the target nucleic acid sequence in order to allow the 3′ target specific portion of the Excess primer to hybridizes to and extend along each previously generated Super-Selective-Primer Strands. In some embodiments, the annealing temperature can be raised again to 70-75° C., permitting hybridization and extension of the full length SuperSelective primer and the full length reverse primer.

In some embodiments, certain temperature sensitive amplification reagents are included in the reaction mixture. Such reagents are described, for example, in the following patents and patent applications: U.S. Pat. No. 7,517,977; U.S. patent application publication No. 2012/0088275; and U.S. provisional patent application No. 61/755,872, each of which are herein incorporated by reference in its entirety.

In some embodiments, the combination of primers and reagents described herein favor amplification of any designated target sequence over its allelic variants, when such variations lie in the sequence complementary to the foot of the SuperSelective Primer. For example, one class of target/variant sequences are base-pair variations resulting from bisulfite conversion which reflect the presence/absence of methylate and unmethylated cytosines in the original target sequence to which the foot of the SuperSelective primer may bind. Prior to selective amplification of such sequences all unmethylated cytosine nucleotides are converted to uridine nucleotides by bisulfite treatment prior to amplification.

### Optimization of LATE-PCR and LEL-PCR Amplification and Multiplexing

In some embodiments, primer sets are optimized for specificity and efficiency in monoplex LATE-PCR or LEL-PCR reactions using SYBR-Green before being integrated into the multiplex assay. In some embodiments the reproducibility and specificity of such reactions is enhanced by addition of one or more additive reagents (e.g., reagents described in U.S. Pat. No. 7,517,977, hereby incorporated by reference in its entirety) that increase polymerase selectivity and thereby suppress non-specific amplification and enhance multiplexing of primer pares (e.g., as described in Rice et al., Nat Protoc 2:2429-2438 (2007), hereby incorporated by reference in its entirety).

### Detection and Analysis of LATE-PCR and LEL-PCR Products

LATE-PCR and LEL-PCR make it possible to generate relatively short or relatively long single-stranded amplicons which can then be scanned for sequence variations using, for example, one or more pairs of Lights-On/Lights-Off probes that are fluorescently labeled in one or more colors, Lights-Off Only probes in combination with a ds-DNA dye and/or by sequencing of amplification the amplification product.

Lights-On/Lights-Off probes are a pair of probes, as well as sets comprised of two or more pairs of probes that hybridize to adjacent nucleic acid sequences on a single-stranded DNA target, such as that produced by LATE-PCR or LEL-PCR amplification. The single-stranded DNA targets can include one or more targets generated in a LATE-PCR or LEL-PCR reaction. In some embodiments the informative sequence within each such target is hybridized to one or more pairs of Lights-On/Lights-Off probes. Each “Lights-On” probe is labeled with a fluorophore and a quencher and can be, for example, a molecular beacon with a self-complementary stem capable of base pairing for one or more contiguous complementary nucleotides. Each “Lights-Off” probe is labeled only with a quencher moiety that can absorb energy from the fluorophore of an adjacently hybridized “Lights-On” probe when both are bound to the target. In some embodiments use of Lights-On/Lights-Off probes allow for the detection of single nucleotide sequence differences by monitoring the effect of temperature changes on the fluorescence emissions of a probe/target mixture. Lights-On/Lights-Off probe technology is more fully described in PCT application No. PCT/US10/53569, hereby incorporated by reference in its entirety. The design criteria for pairs of Lights-On/Lights-Off probes are described in Rice et al, Nucleic Acids Research 40:e164 (2012) and PCT application No. PCT/US10/53569, each of which are incorporated by reference in its entirety.

In certain embodiments, sets of Lights-On/Lights-Off probes, (e.g., one for each amplicon), are designed to hybridize to the single-stranded amplicons at the end of a LATE-PCR or LEL-PCR amplification over the same wide temperature range, the temperature range being at least 5° C. below the limiting primer annealing temperature of the reaction. In some embodiments each set is labeled in a different color and each set spans the entire non-primer sequence of the amplicon. In certain embodiments, some probes include nucleotide mismatches to their target sequences to adjust the probe melting temperature. Lights-On probes are labeled with a fluorophore and a quencher at opposite ends. Lights-Off probes labeled with a quencher at the 5′ end are blocked at their 3′ end, for example, with a by covalent linkage of a three carbon, C3, moiety.

In some embodiments, the LATE-PCR or LEL-PCR amplification product is detected and analyzed using Lights-Off Only probes. Analysis of single-stranded amplification products using Lights-Off Only probes is similar to detection using Lights-On/Lights-Off probe sets, except a dsDNA fluorescent dye, such as SYBR® Green, is used in the place of a fluorescently labeled Lights-On probe. Thus, in certain embodiments, single-stranded DNA amplification products are detected using dyes that fluoresce when associated with double strands in combination with one or more hybridization probes that hybridize to a target nucleic acid sequence and that are labeled with a non-fluorescent quencher moiety, for example, a Black Hole quencher (“Lights-Off Only probes”). The fluorescent signature produced by from the dsDNA binding dye as a function of temperature over a temperature range that includes the melting temperature of such hybridization probe or probes is analyzed to detect sequence variations indicating the original methylation state of the target sequence.

In some embodiments the Lights-On probes and/or the Lights-Off probes are designed taking into account constraints imposed by the target sequences and temperature dependent secondary structures of the single-stranded target amplicons. For instance, one or two contiguous probes may bind to a sequence at a temperature that is higher than that needed for the sequence to form a hairpin loop, thereby preventing loop formation when the temperature is lowered. In some embodiments probes are designed to hybridize to target sequences such that they generate the simplest reference fluorescent signature on normal targets to ensure that fluorescent signatures dependent on original DNA methylation status stand out distinctly by comparison to those not altered by original DNA methylation status. Other information with regard to the design of Lights-On/Lights-Off probes are described, for example, in Rice et al., Nucleic Acids Res (2012) and Carver-Brown et al., J Pathog 2012:424808 (2012), each of which is hereby incorporated by reference in its entirety.

In some embodiments multiplex LATE-PCR or LEL-PCR amplification is carried out according to standard LATE-PCR conditions (e.g., 25 μl reactions consisting of 1× Platinum Taq buffer, 3 mM MgCl2, 400 μM of each deoxynucleotide triphosphate, 50 nM of each limiting primer, 1 μM of each excess primer, 100 nM of each Lights-On probe, 300 nM of each Lights-Off probe, 2.0 units of Platinum Taq DNA polymerase, and bisulfite-treated genomic DNA). In some embodiments the concentration of each Lights-On probe is slightly less than the anticipated maximal yield of single-stranded DNA amplicons generated in the LATE-PCR to guarantee complete binding of all the Lights-On probes and minimize differences among replicate fluorescent signatures (e.g., 100 nM if the above amplification conditions are used). In some embodiments the concentration of each Lights-Off probe is set three-fold higher than the concentration of each Lights-On probe to guarantee that every bound Lights-On probe will have a Lights-Off probe hybridized next to it at low temperature (e.g., 300 nM if the above conditions are used). In some embodiments amplification is carried out for at least 30, 35, 40, 45, 50, 55, 60, 65, 70, 75 or 80 cycles.

In some embodiments at least one non-amplifiable pair of oligonucleotides, comprised of a fluorescently labeled oligonucleotide and an excess of a complementary oligonucleotide labeled with a moiety that quenches fluorescence (such as a Black Hole Quencher) is added to the amplification reaction. The melting temperature of the non-amplifiable pair of oligonucleotides is higher than the melting temperature of all probe-target hybrids in the reaction. The pair of non-amplifiable oligonucleotides therefore serves as an internal temperature mark may also serve to enhance polymerase selectivity as described in U.S. Provisional Pat. App. No. 61/755,872, hereby incorporated by reference in its entirety.

In certain embodiments, endpoint analysis is used to determine the original methylation status of the one or more CpGs of interest. In some embodiments this endpoint analysis comprises a step of cooling the reaction from a point above the melting temperature of the Lights-On/Lights-Off probes to a temperature below the melting temperature of the Lights-On/Lights-Off probes to allow hybridization of the Lights-On/Lights-Off probes. For example, in some embodiments the temperature is cooled from 85° C. to 5° C. at 1° C. degree decrements every 30 seconds to allow for hybridization of the Lights-On/Lights-Off probes. In some embodiments the endpoint analysis comprises a step of maintaining the temperature of the reaction at a temperature below the melting temperature of the Lights-On/Lights-Off probes for a period of time sufficient for the probe binding to equilibrate. For example, in some embodiments the temperature of the reaction is maintained at the lowest temperature used for analysis for 10 minutes. In some embodiments the endpoint analysis comprises the step of heating the reaction from a point below the melting temperature of the Lights-On/Lights-Off probes to a temperature above the melting temperature of the Lights-On/Lights-Off probes to allow melting of the Lights-On/Lights-Off probes, during which time the fluorescence emitted by the reaction is monitored. For example, in some embodiments the reaction is then heated from 5° C. to 85° C. in 1° C. increments every 30 second with fluorescent acquisition at every degree.

In some embodiments fluorescent signatures are obtained by plotting the negative first derivative of the raw fluorescent signals from the melting analysis relative to temperature (−dF/dT) as a function of temperature without any background signal subtraction or data normalization. In some embodiments DNA methylation events are identified by comparing the shape of fluorescent signature from the matching untreated DNA control to the shape of the fluorescent signature from the bisulfite-treated sample. Differences in the shape due to temperature shifts in the position of the peaks and valleys of the fluorescent signatures indicate that the DNA methylation status does not match the status assumed by the probes used. For example if the probe is designed to be complementary to a CpG sequence at a particular CpG, then a change in fluorescent signature indicates that the dinucleotide under examination is no longer CpG, the change being indicative of the fact that the dinucleotide was originally an unmethylated CpG. If the probe is designed to be complementary to a TpG sequence at the position of the dinucleotide under examination, then a change in fluorescent signature indicates that the CpG was originally methylated. According to the method of this analysis, differences in fluorescent signature intensities without a temperature shift reflected differences in amplicon yield rather than a change in nucleotide sequence due to methylation.

CpG dinucleotides are often, but not exclusively, found within CpG islands in which many CpG dinucleotides share a common methylation state. Thus, in certain embodiments, LATE-PCR assays described herein are designed to measure the methylation state of a particular CpG dinucleotide (referred to herein as “CpG(1) assays”). In certain embodiments, LATE-PCR or LEL-PCR assays described herein are designed to measure the methylation state of multiple CpG dinucleotides (referred to herein as “CpG(1+) assays”), for example, within a CpG island.

In certain embodiments, CpG(1) assays are performed. In some embodiments such assays using sequence Low-Tm or Superlow-Tm probes or a single-pair of Lights-On/Lights-Off probes for analysis of the original methylation status of a particular CpG dinucleotide. In some embodiments CpG(1) assays include analysis of more than one CpG dinucleotide in one or more amplicons, with each targeted CpG dinucleotide analyzed using sequence Low-Tm or Superlow-Tm probes or pairs of Lights-On/Lights-Off probes having a different fluorescent color. The resulting set of fluorescent signatures for original methylation status at the different CpG dinucleotides will depend on a number of variables, including: 1) whether the CpG dinucleotide at a particular site was located under a Lights-On probe or a Lights-Off probe; 2) whether the probe that hybridizes at that site is complementary to the G or the A nucleotide generated from the original CpG site; and 3) whether the melting temperature (Tm) of the Lights-Off probe is lower than, equal to, higher than the Tm of the Lights-On probe as a function of the original methylation status of the CpG at that site. In certain embodiments, a CpG(1) assay will establish which of the possible methylation states (methylated, hemi-methylated or unmethylated) was originally present at a targeted CpG site.

In some embodiments a CpG(1+) assay is performed in which sets of Lights-On/Lights-Off probes comprised of pairs of probes labeled in the same color are used to scan amplicons containing two or more CpG dinucleotides. Complete resolution of all possible methylation states of a first CpG dinucleotide and a second CpG dinucleotide targeted in a CpG(1+) assay can be achieved, for example, by separating the temperature range at which the probes for the first CpG site is analyzed from the temperature range at which the probes, in the same color, are used to analyze a second CpG site. The resulting fluorescent contour and/or fluorescent signature is composite of all Lights-On/Lights-Off probe signals in that particular color. Composite patterns of this nature of are highly reproducible and highly responsive to even single nucleotide changes anywhere in the target and therefore enable detection of any change in methylation status in the original target sequence. Methylation status can be confirmed by sequencing the amplicon (e.g., by Dilute-‘N’-Go sequencing), as described below.

Because many methylation/demethylation sites within a methylation island undergo changes in concert with each other, certain embodiments utilize high specificity primers that allow for preferential amplification of a particular DNA strand based on its original methylation status. Such high specificity primers serve as the Limiting primer in CpG(1+) assays. Examples of such high specificity primers include: ARMS primers (described in, for example, Newton et al., Nucleic Acids Res. 17:2503-2516 (1989), which is hereby incorporated by reference), DPO primers (Chun et al., Nucleic Acids Res., 35:e40 (2007), which is hereby incorporated by reference), MyT primers (described in, for example, U.S. Patent Application Publication No. US/2011/0129832, which is hereby incorporated by reference), and SuperSelective Primers, as recently described by the laboratory of Dr. Fred Kramer at the Public Health Research Institute. Additional reagents, such as those described in international patent application WO/2006/044995, international patent application WO/2010/105074, and U.S. provisional patent application 61/755,872, each of which is hereby incorporated by reference, can also be added to reactions to enhance polymerase selectivity. One manifestation of polymerase selectivity is increased primer specific amplification of a particular strand on the basis of a single nucleotide mismatch.

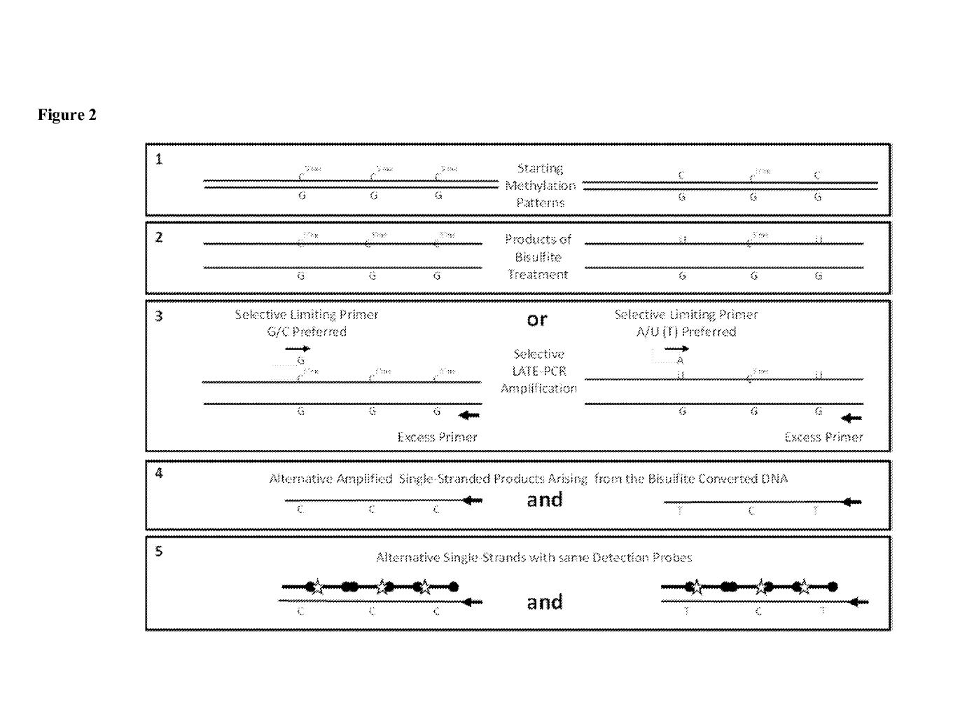

As shown in FIG. 2, in some embodiments bisulfite conversion, target amplification, and product analysis are carried out in steps. All of these steps can be carried out in a single-tube, or, bisulfite conversion of the target genome can occur in a separate vessel from that used for target amplification and product analysis. Target amplification and analysis can take place as a closed-tube homogeneous LATE-PCR or LEL-PCR amplification reaction followed by end-point analysis of the single-stranded DNA product using, for example, either Lights-On/Lights-Off probes or Lights-Off only probes to analyze the nucleotide composition of the target. The amplification reaction begins with a sequence selective limiting primer that can be designed to start DNA synthesis by preferentially hybridizing to a sequence containing either a G/C nucleotide or an A/U(T) nucleotide in a position that corresponds to a CpG dinucleotide in the original target sequence. As a result, there is either a C or a T nucleotide near the 3′ end of the single-stranded product of LATE-PCR or LEL-PCR amplification (i.e. the sequence that is complementary to the particular sequence selective limiting primer used). Further toward the 5′ end of each product are additional sites that reflect the original methylation status of other CpG dinucleotides in the amplified target. The sequences of these sites are accessed via probed/target end-point detection using the same set of Lights-On/Lights-Off probes or the same set of Lights-Off Only probes.

In addition, in some embodiments blockers are used that bind to their targets in an allele specific manner and selectively prevent primer extension. Some blockers, such as those made of LNA's and PNA's are located downstream of the 3′ end of the primer. Other blockers overlap with the 3′ end of the primer. Such approaches can be adapted for use with LATE-PCR by designing the Limiting primer to be a selective primer and the excess primer to be a non-selective primer, as depicted in FIG. 2. In the case of methylation islands, the specific methylation-dependent single-stranded product amplified in the reactions may include dinucleotides whose sequence reflects the original methylation status of additional CpG dinucleotides in the CpG island being targeted. In such cases, sets of Lights-On/Lights-Off probes can be used to assess these additional CpG dinucleotides (FIG. 2).

Additional description of such approaches is provided in U.S. Pat. App. Pub. No. 2014/0024033, which is hereby incorporated by reference.

Because many methylation/demethylation sites within a methylation island undergo changes in concert with each other, in certain embodiments high specificity primers are used that allow for preferential amplification of a particular DNA strand based on its original methylation status. In these embodiments the high specificity primers serve as the Limiting primer in CpG(1+) assays. Examples of such high specificity primers are described, for example, in U.S. Pat. No. 6,365,729, incorporated by reference herein in its entirety. The specific methylation-dependent single-stranded product amplified in the reactions may include dinucleotides whose sequence reflects the original methylation status of additional CpG dinucleotides in the CpG island being targeted. In such cases, sets of Lights-On/Lights-Off probes can be used to assess these additional CpG dinucleotides.

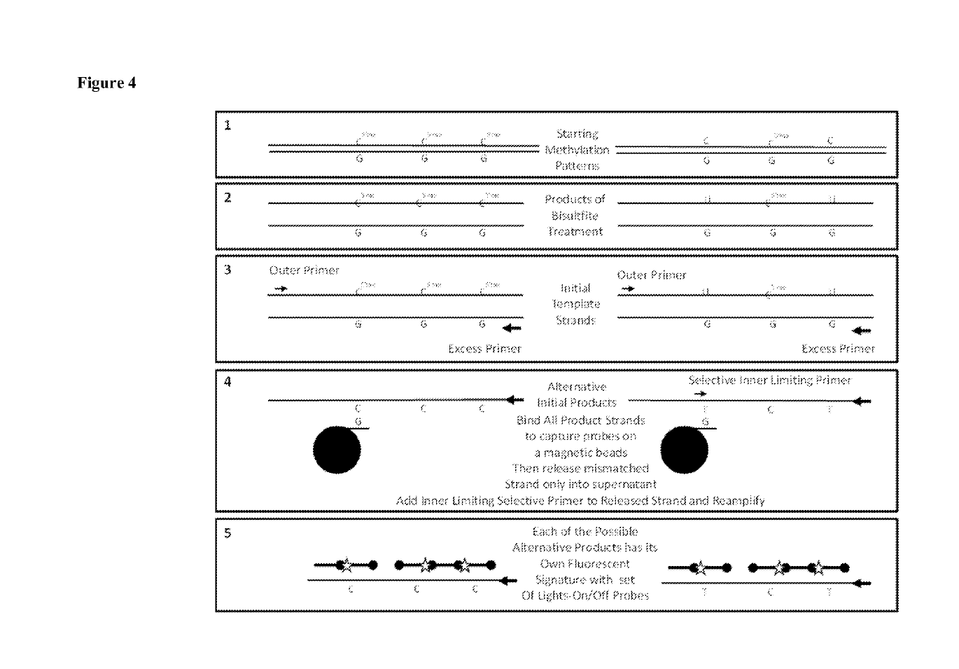

Moreover, because many methylation/demethylation sites within a methylation island undergo changes in concert with each other, additional non-primer methods can be utilized to preferentially amplify or purify a particular DNA strand based on its original methylation status. One such example known as DISSECT is depicted in FIG. 4 and described in, for example, Guha et al., Nucleic Acids Research 41:e50 (2013), incorporated by reference herein in its entirety. The specific methylation-dependent single-stranded prepared in this way may include additional dinucleotides whose sequence reflects their original methylation status in the CpG island being targeted. In such cases, sets of Lights-On/Lights-Off probes can be used to assess these additional CpG dinucleotides.

As shown in FIG. 4, in some embodiment's bisulfite conversion, initial target amplification, and removal of initial products, secondary amplification and end-product analysis are carried out in steps. Steps 1-3 can be carried out in a single-tube, or, bisulfite conversion of the target genome can occur in a separate vessel from that used for initial target amplification and removal of initial products. Removal of initial products can be accomplished through a temperature dependent step in which a sequence-variant specific oligonucleotide probe that has been immobilized on a solid support (e.g., a magnetic bead) is used to selectively bind to and remove from solution nucleic acids containing the sequence variant to which it preferentially hybridizes while leaving other variants in solution. This step can be repeated and the oligonucleotide on the capture probe can be targeted to any discerning sequence in the target strands. The final product of the selection process is one or more molecules of the target used for re-amplification using a second limiting primer and either the same or a different excess primer. These primers are LATE-PCR primers which generate a single-stranded DNA product. Target re-amplification and analysis take place in a closed-tube homogeneous LATE-PCR or LEL-PCR amplification reaction followed by end-point analysis of the single-stranded DNA product using either Lights-On/Lights-Off probes or Lights-Off only probes to analyze the nucleotide composition of the target. Each product has sites that reflect the original methylation status of CpG dinucleotides in the amplified target. The sequences of these sites are determined via probed/target end-point detection using the same set of Lights-On/Lights-Off probes or the same set of Lights-Off Only probes.

As one skilled in the art will appreciate the selective primer approach described above and in FIG. 2 can be combined with the selective magnetic bead approach above and in FIG. 4. In one instance the reaction can begin as described in FIG. 4 but can proceed as described in FIG. 2 but making the Inner Limiting Primer into a selective primer. In another instance, the reaction can begin as described in FIG. 2 with preferential amplification of one sequence variant, followed by magnetic bead removal of the undesired variant or variants, followed by re-amplification of the desired variant using an Inner Limiting Primer.

In certain embodiments, the methylation state of the LATE-PCR or LEL-PCR amplification product is detected and/or confirmed by sequencing the amplification product. In some embodiments the amplification product is sequenced directly. In some embodiments the amplification product is first cloned in a vector (e.g., using TOPO/TA cloning) and then the resulting cloned plasmid is sequenced. Any sequencing method can be used. In certain embodiments chain termination sequencing, sequencing by ligation, sequencing by synthesis, pyrosequencing, ion semiconductor sequencing, single-molecule real-time sequencing, dilute-‘n’-go sequencing and/or 454 sequencing is used. In certain embodiments, amplification products are sequenced using a Dilute-‘N’-Go method. The Dilute-‘N’-Go method is described in Rice et al., Nat Protoc 2:2429-2438 (2007) and Jia et al., Nucleic Acids Res 38:e119 (2010), each of which is hereby incorporated by reference in its entirety). When the amplification product is sequenced, all the cytosine residues remaining in the sequence represent cytosine residues that were methylated in the genome.

### EXAMPLES

### Example 1

### Allelic-Discrimination Using SuperSelective Primers

SuperSelective primers and their corresponding reverse primers were used to illustrate in two separate PCR assays the preferential amplification of target sequences in which the 3′ end of the SuperSelective primers formed fully complementary hybrids (matched targets) versus target sequences in which the 3′ end of the SuperSelective primers formed mismatched hybrids (mismatched targets). The first PCR assay selectively amplified the single nucleotide L858R mutation within the human epidermal growth factor receptor (EGFR) gene that confers sensitivity to the tyrosine kinase inhibitors such as gefitinib. The second PCR assay selectively amplified the single nucleotide V600E mutation within the human B-RAF gene that confers sensitivity to the tyrosine kinase inhibitor vemurafenib.

The sequence of the SuperSelective Primers and their corresponding reverse primers for the two PCR assays were as follows:

Forward EGFR SuperSelective primer complementary
to the L858R mutation:
[SEQ 1]
5′ CTGGTGAAAACACCGCAGCATGTCGCACGAGTGAGCCCTGGGCGG 3′

where the sequence preceding the underlined sequence corresponds to the anchor sequence, the underlined sequence corresponds to the bridge sequence, the sequence following the underlined sequence corresponds to the foot sequence, and the nucleotide shown in bold corresponds to the nucleotide that is matched the to the EGFR L858R mutation and mismatched to the wild-type sequence. The melting temperature of the EGFR anchor sequence is 71° C.

Reverse EGFR primer:
[SEQ 2]
5′ GCATGGTATTCTTTCTCTTCCGCA 3′

which has a Tm of 60° C.

Forward BRAF SuperSelective primer complementary
to the L858R mutation:
[SEQ 3]
5′ AGACAACTGTTCAAACTGATGGGAAAACACAATCATCTATTTCTC 3′

where the sequence preceding the underlined sequence corresponds to the anchor sequence, the underlined sequence corresponds to the bridge sequence, the sequence following the underlined sequence corresponds to the foot sequence, and the nucleotide shown in bold corresponds to the nucleotide that is matched the to the BRAF V600E mutation and mismatched to the wild-type sequence. The melting temperature of the BRAF anchor sequence is 71° C.

Reverse B-RAF primer:
[SEQ 4]
5′ AGACAACTGTTCAAACTGATGGGA 3′

which has a Tm of 60° C.

The DNA targets were located on plasmids containing in which a 115-base pair gene fragment from EGFR exon 21 containing the L858R mutant sequence, the corresponding EGFR wild-type sequence, a 116-base pair fragment B-RAF V600E mutant sequence, or the corresponding B-RAF wild-type sequence, were inserted into a pGEM-11Zf(+) plasmid. These plasmids were digested with the endonuclease MseI (New England Biolabs). Prior to the use with SuperSelective primers, corresponding pairs of mutant and wildtype targets were matched in concentration to 10,000 copies/μl each by dilution in 10 mM Tris-Cl, pH 8.3. Equimolar primer concentrations were confirmed in separate reactions after real-time amplification with SYBR Green using the following primers that do not overlap with the mutations together with their corresponding reverse primer:

EGFR forward anchor:
[SEQ 5]
5′ CTGGTGAAAACACCGCAGCATGTC 3′
BRAF forward anchor:
[SEQ 6]
5′ AGACAACTGTTCAAACTGATGGGA 3′

Amplifications were carried out in replicate reactions in a Stratagene MX3005P thermal cycler (Agilent, Santa Clara, Calif.). PCR assays were performed in a 30 μl volume containing of 1× Platinum Taq buffer (Invitrogen, Carlsbad, Calif.), 3 mM MgCl2, 250 μM of each deoxynucleotide triphosphate, 60 nM of forward SuperSelective primer, 60 nM of reverse primer, 0.24×SYBR Green (Invitrogen, Carlsbad, Calif.), 1.5 units of Platinum Taq DNA polymerase (Invitrogen, Carlsbad, Calif.), and 10,000 copies of either mutant or wild-type plasmids. The reactions were first incubated at 95° C. for three minutes, followed by 60 cycles of denaturation at 95° C. for 15 seconds, primer annealing at 60° C. for 15 seconds, and primer extension at 72° C. for 30 seconds. SYBR Green fluorescent intensity was measured during each extension step throughout the course of each reaction.

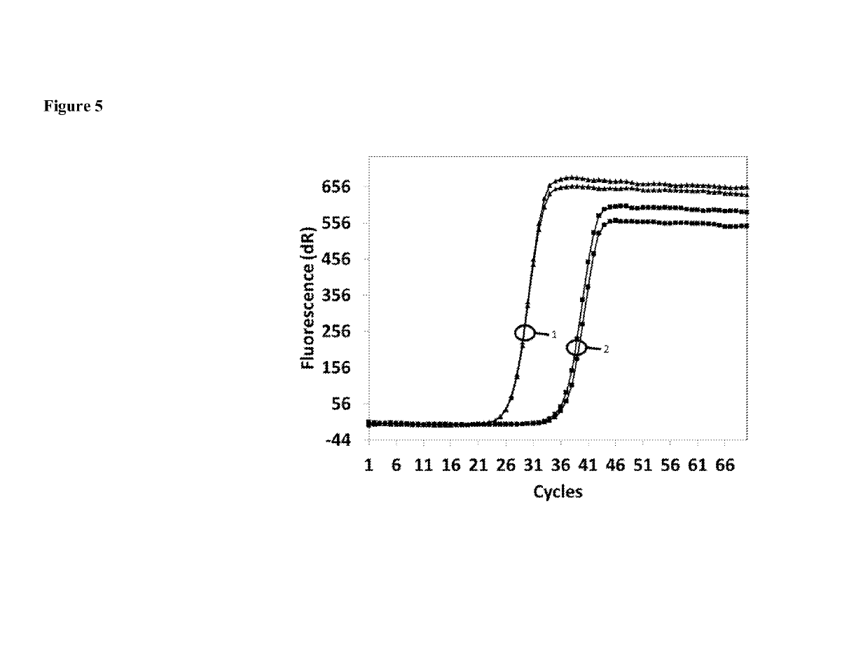

FIG. 5 shows preferential amplification of 10,000 copies of mutant EGFR L858R DNA (Curve 1) relative to 10,000 copies of wild-type EGFR DNA (Curve 2).

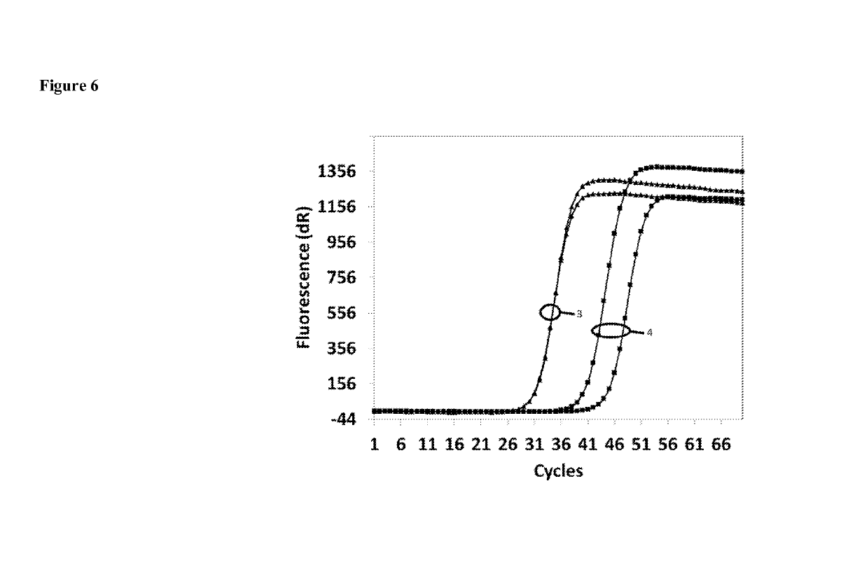

FIG. 6 shows preferential amplification of 10,000 copies of mutant BRAF V600E DNA (Curve 3) relative to 10,000 copies of wild-type BRAF DNA (Curve 4).

### Example 2

### Increasing Concentrations of Temperature-Dependent Reagent EP003 Enhances Allele Discrimination of SuperSelective Primers

Temperature-Dependent Reagents is a category of terminally-modified, double-stranded DNA additives that, depending on the configuration of the modifications at the end of the strands and the concentration of the reagent, determine the selectivity/specificity and/or activity of Taq DNA polymerase in PCR amplification reactions in a temperature-dependent manner (e.g., as described in U.S. patent application publication No. 2012/0088275, which is hereby incorporated by reference). The temperature dependency is due to fact that Temperature-Dependent Reagent is only active at temperatures where it remains double-stranded. This class of reagents make it possible to define the stringency of the reaction not only by controlling the temperature of the assay in a precise manner, as in conventional PCR, but also by allowing the reaction to cool down to within a range of temperatures where the reagent becomes double stranded.

As described herein, Temperature-Dependent Reagent EP003 improves the selectivity of SuperSelective primers without altering the temperature of the annealing or extension steps of the reaction. The composition of double stranded Temperature-Dependent Reagent EP003 is as follows:

[SEQ 7]
5′ GGAGCAAAATAGCAATGAGGTA 3′[Dabcyl-Q]
3′ [Dabcyl-Q]CGTTTT ATC GTTACT CAT [5-Dabcyl] 5′

The resulting EP003 hybrid has a melting temperature of 63.1° C. at a concentration of 100 nM.

The same amplification reactions described in Example 1 were carried out in the presence of increasing concentrations of EP003 (0 nM, 25 nM, 50 nM and 100 nM). Amplifications were done in replicate reactions in a Stratagene MX3005P thermal cycler (Agilent, Santa Clara, Calif.). PCR assays were performed in a 30 μl volume containing 1× Platinum Taq buffer (Invitrogen, Carlsbad, Calif.), 3 mM MgCl2, 250 μM of each deoxynucleotide triphosphate, 60 nM of forward SuperSelective primer, 60 nM for reverse primer, 0.24×SYBR Green (Invitrogen, Carlsbad, Calif.), 0-100 nM EP003, 1.5 units of Platinum Taq DNA polymerase (Invitrogen, Carlsbad, Calif.), and 10,000 copies of either mutant or wild-type plasmids. The reactions were first incubated at 95° C. for three minutes, followed by 60 cycles of denaturation at 95° C. for 15 seconds, primer annealing at 60° C. for 15 seconds, and primer extension at 72° C. for 30 seconds. SYBR Green fluorescent intensity was measured during each extension step throughout the course of each reaction.

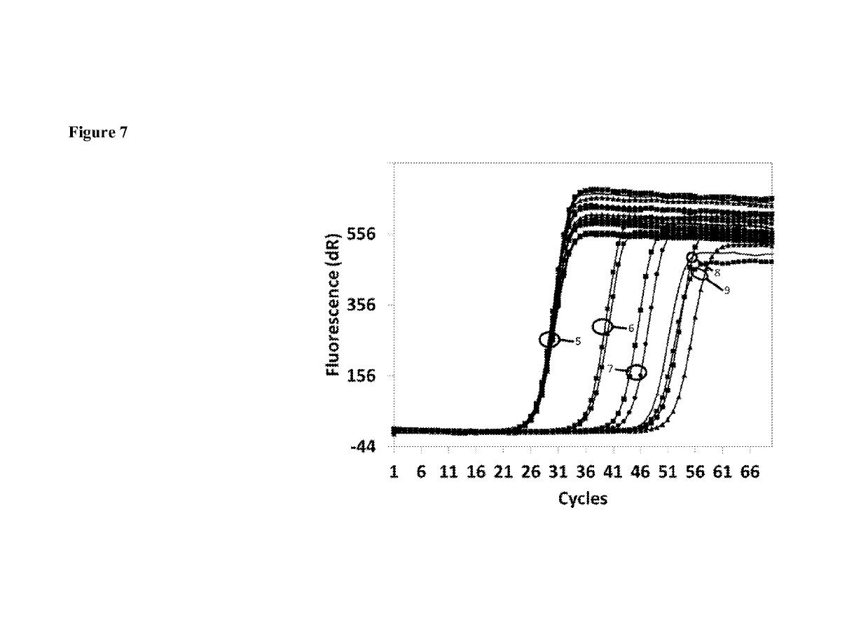

FIG. 7 shows that increasing concentrations of EP003 did not appreciably affect amplification of EGFR L858R mutant targets (Curve 5, all EP003 concentrations) but preferentially delayed the amplification of wild-type EGFR targets (Curve 6, 0 nM EP003; Curve 7, 25 nM EP003; Curve 8, 50 nM EP003; Curve 9, 100 nM EP003).

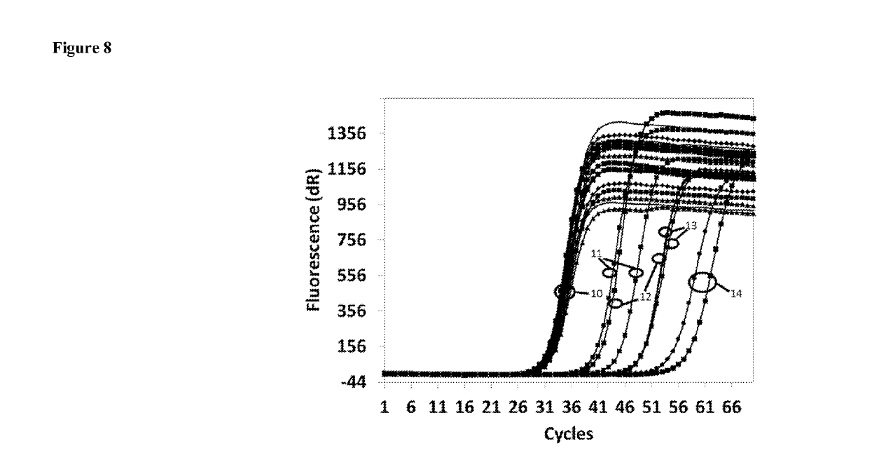

FIG. 8 shows that increasing concentrations of EP003 did not appreciably affect amplification of BRAF V600E mutant targets (Curve 10, all EP003 concentrations) but preferentially delayed the amplification of wild-type BRAF targets (Curve 11, 0 nM EP003; Curve 12, 25 nM EP003; Curve 13, 50 nM EP003; Curve 14, 100 nM EP003).

Temperature-Dependent Reagent EP003 can be combined with other Temperature-Dependent Reagents with various configurations of terminal modifications (e.g., as described in U.S. Patent application publication No. 2012/0088275) to further improve primer specificity/selectivity.

### Example 3

### SuperSelective Primers in LATE-PCR and LEL-PCR Reactions

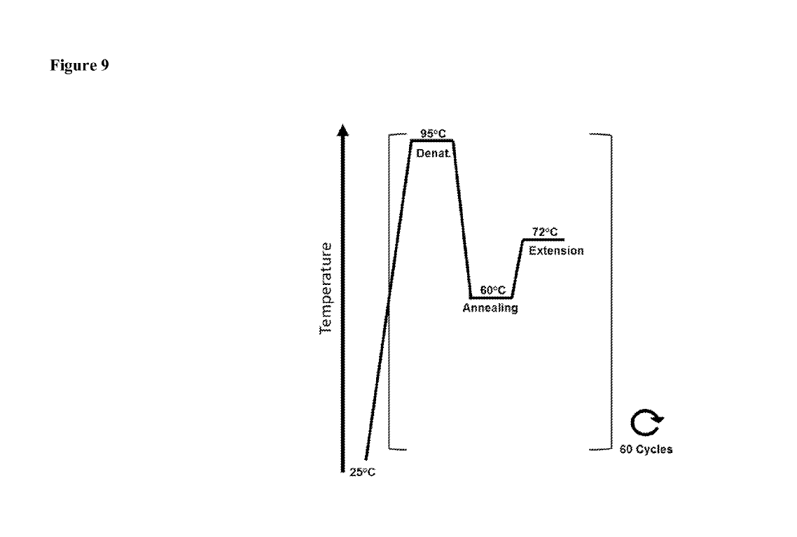

Mutant templates are distinguished from wild-type templates when SuperSelective primers hybridize to target molecules in the original sample. When SuperSelective primers overlap the sequence difference between mutant and wild-type, amplicons resulting from mispriming on wild-type targets are identical to amplicons from mutant targets. It would therefore be desirable to restrict the number of thermal cycles in which the SuperSelective primers hybridize to the original wild-type targets to minimize the possibility of unintended initiation events on wild-type targets. It would also be desirable to increase the stringency of hybridization of the SuperSelective primers in order to minimize unintended extension on wild-type targets. The temperature cycling profile used in conventional SuperSelective primer based PCR does not provide such flexibility (i.e., since the melting temperatures of the anchor sequence of the SuperSelective primer and the reverse primers typically are 71° C. and 60° C., respectively, any attempt to increase the stringency of the SuperSelective primers by raising the annealing temperature above 60° C. would reduce the number of reverse primers participating in the reaction and result in lower amplification efficiency, FIG. 9).

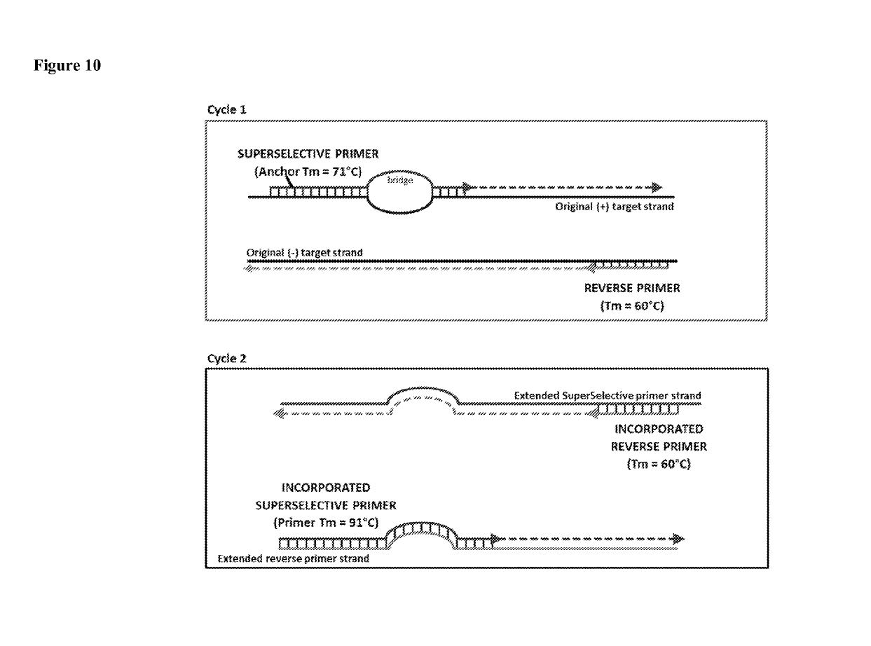

A feature of certain SuperSelective primers is that the 5′-anchor sequence is linked to the 3′-foot sequence by a 14-nucleotide long bridge sequence that is not complementary to the target sequence. As a result, when the primer is hybridized to an original target molecule, the bridge sequence in the primer remains single stranded and forms a bubble. Once a SuperSelective primers are successfully extended on the matched target sequence, however, the amplicons produced in subsequent amplification cycles incorporate the entire SuperSelective primer sequence (including the bridge sequence) and the melting temperature of the SuperSelective primer on the now fully complementary amplicon increases (FIG. 10).

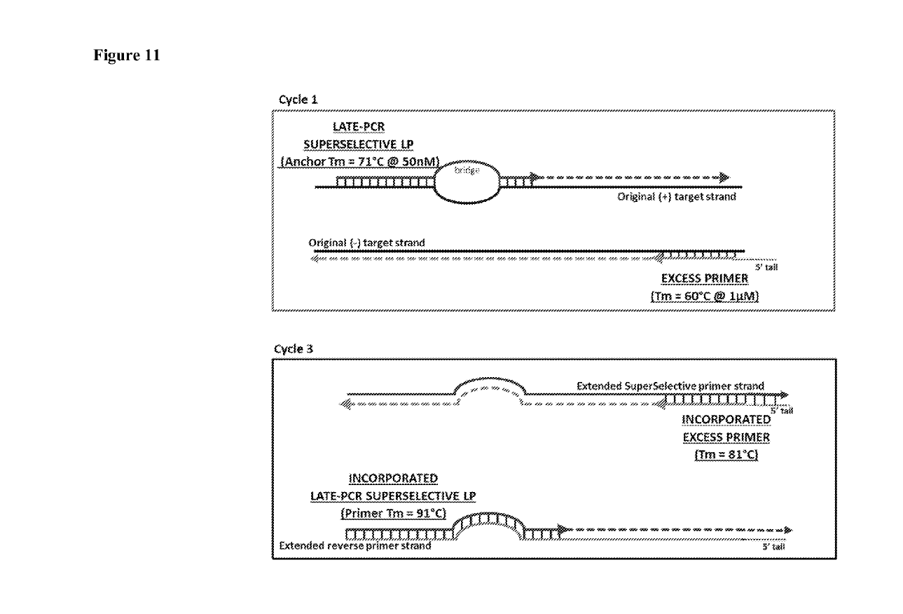

By designing the reverse primer such that its Tm also increases during amplification, it is possible to restrict the number of thermal cycles where the SuperSelective primers hybridize to the original target nucleic acid sequence by raising the annealing temperature of the reaction to a temperature where only fully complementary primers bound to amplicon targets participate in the reaction. To accomplish this, the SuperSelective primer and the reverse primer were converted to LATE-PCR primers and a 5′ tail non-complementary to the original target sequence was added to the LATE-PCR reverse primer such that once incorporated into an amplicon, the Tm of the fully complementary reverse primer on the amplicons targets increased after the first cycle of amplification (FIG. 11).

To convert conventional primer pairs to LATE-PCR primers, the SuperSelective primer was used as the limiting primer at 50 nM without any modification. The length of the reverse primer including addition of a 5′ tail non-complementary to the original target sequence was adjusted to allow this primer to be used as an excess primer at 1000 nM while keeping the concentration-adjusted Tm at 60° C.

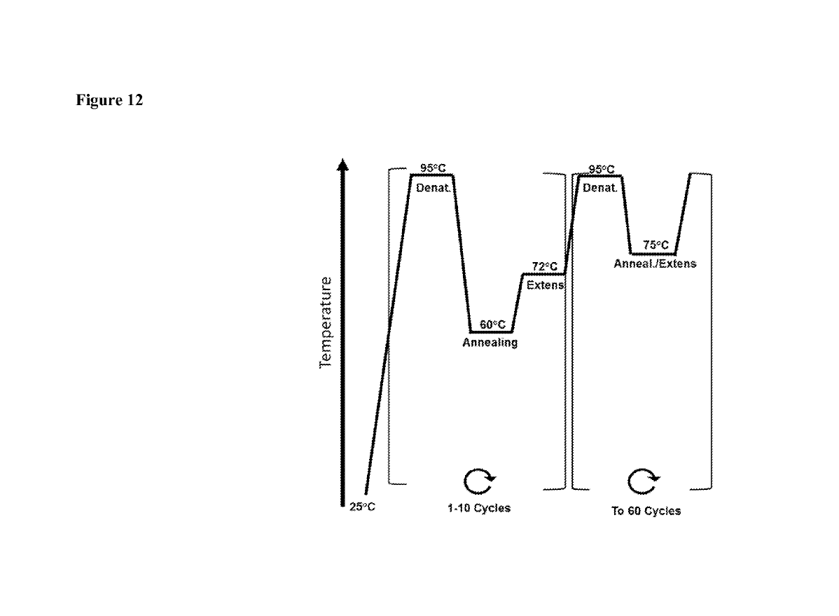

The LATE-PCR primer design allows a different temperature cycling profile to be used with SuperSelective primers (e.g., as depicted in FIG. 12). In this scheme, the original target molecules are interrogated with SuperSelective primers for selective amplification of mutant targets in the first 1-10 amplification cycles. During these initial cycles, the excess primers do not meet LATE-PCR design criteria, since the Tm of these primers (60° C.) is more than 20° C. below the amplicon Tm (LATE-PCR design criteria generally specifies that the excess primer Tm should be less than 20° C. the amplicon Tm). After these initial selective cycles, the annealing temperature is increased to 75 C to allow exclusive exponential amplification of amplicon targets without any further interrogation of original target molecules.

By design, the Tm of the anchor sequence of the SuperSelective primer is >10° C. above the excess primer Tm (71° C. compared to 60° C.). This allows the implementation of a PCR approach wherein the SuperSelective primer undergoes several rounds of linear amplification at an annealing temperature of 71° C. to interrogate mutant and wild-type targets under stringent conditions. The temperature is then dropped once to 60° C. to enable hybridization and extension of the excess primer for one cycle. This is followed by multiple round of temperature cycles with an annealing temperature of 75° C. to enable exclusive amplification of amplicon targets by LATE-PCR. This approach where linear amplification of a primer is followed by exponential amplification and linear amplification under LATE-PCR conditions is called Linear-Exponential-Linear (LEL) PCR (FIG. 13).

To show proof-of-principle of LEL-PCR, the following LATE-PCR SuperSelective primer and reverse excess primers were used:

LATE-PCR SuperSelective Limiting Primer:
[SEQ 8]
5′ CTGGTGAAAACACCGCAGCATGTCGCCCGAGTGAGCCCTGGGCAG 3′

where the sequence preceding the underlined sequence corresponds to the anchor sequence (24 nucleotides), the underlined sequence corresponds to the bridge sequence, the sequence following the underlined sequence corresponds to the foot sequence, and the nucleotide shown in bold corresponds to the nucleotide that is matched the a T nucleotide on the matched original target sequence that simulates bisulfite conversion of a unmethylated cytosine of a CpG dinucleotide. The concentration-adjusted Tm of the anchor sequence is 71° C. at 50 nM.

LATE-PCR Excess reverse primer with a 5′ non-
complementary tail:
[SEQ 9]
5′ GAGGAATGAAACGGAGGAAGACGTACGTATTCTTTCTCTTCCGCA 3′

where the underlined sequence corresponds to the 5′ tail non-complementary to the original target sequence. The concentration-adjusted Tm of the this primer is 60° C. at 1000 nM.

The original targets for these primers consisted of the following double-stranded synthetic oligonucleotides (IDT, Coralville, Iowa):

Matched simulated unmethylated target:
[SEQ 10]
5′CTAAAGCCACCTCCTTACTTTGCCTCCTTCTGCATGGTATTCTTTCTC
TTCCGCATGCACCAGTTTGGCCTGCCCAAAATCTGTGATCTTGACATGCT
GCGGTGTTTTCACCAGTACGTTCCTGG3′

where the foot region of the LATE-PCR SuperSelective primer is fully complementary at the T nucleotide shown in bold that simulates bisulfite conversion of a unmethylated cytosine of a CpG dinucleotide.

Mismatched simulated methylated target:
[SEQ 11]
5′CTAAAGCCACCTCCTTACTTTGCCTCCTTCTGCATGGTATTCTTTCTC
TTCCGCATGCACCAGTTCGGCCCGCCCAAAATCTGTGATCTTGACATGCT
GCGGTGTTTTCACCAGTACGTTCCTGG3′

where the foot region of the LATE-PCR SuperSelective primer is mismatched at the C nucleotide shown in bold that simulates a methylated cytosine.

Amplifications were carried out in replicate reactions in a Stratagene MX3005P thermal cycler (Agilent, Santa Clara, Calif.). PCR assays were performed in a 15 μl volume consisting of 1× Platinum Taq buffer (Invitrogen, Carlsbad, Calif.), 3 mM MgCl2, 250 μM of each deoxynucleotide triphosphate, 50 nM of LATE-PCR SuperSelective primer, 1000 nM for LATE-PCR reverse primer, 0.24×SYBR Green (Invitrogen, Carlsbad, Calif.), 1.5 units of Platinum Taq DNA polymerase (Invitrogen, Carlsbad, Calif.), and 10,000 copies of either matched un-methylated target or matched methylated target. The reactions were first incubated at 95° C. for three minutes, followed by 1-10 cycles of denaturation at 95° C. for 15 seconds and primer annealing at 70° C. for 15 seconds, and primer extension at 80° C. for 30 seconds (note: primer extension was done at 80° C. instead of the customary 72° C.-75° C. to prevent further hybridization events of the SuperSelective primer during extension), one cycle of denaturation at 95° C. for 15 seconds and primer annealing at 60° C. for 15 seconds, and primer extension at 72° C. for 30 seconds, and 50 cycles of 95° C. for 15 seconds and primer annealing/extension at 75° C. for 30 seconds. SYBR Green fluorescent intensity was measured during each extension step throughout the course of each reaction.

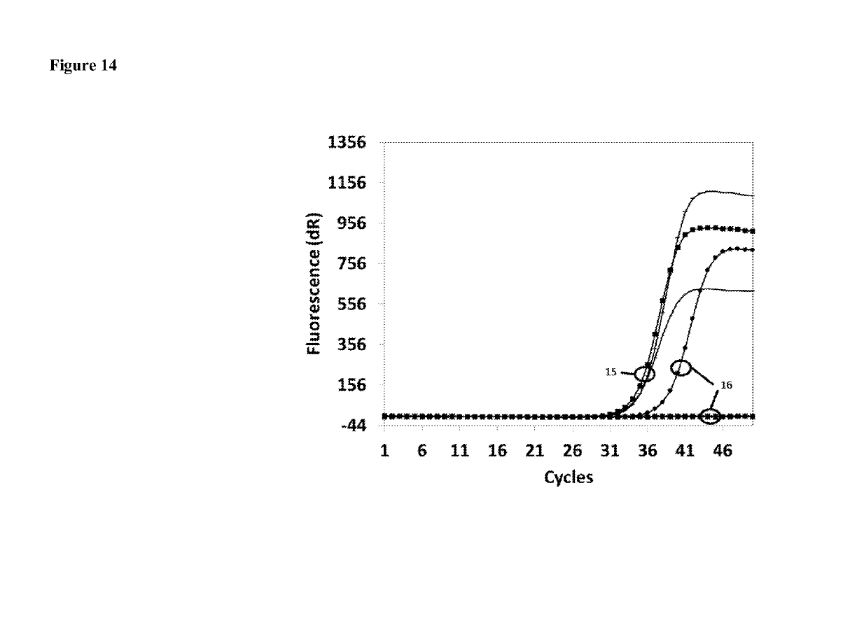

FIG. 14 shows preferential amplification of three replicates of 10,000 copies of matched unmethylated targets (Curves 15) relative to only one out of three replicates of 10,000 copies of mismatched methylated targets (Curves 16) after a single round of linear extension of the limiting SuperSelective primer at 70° C. This result demonstrates that it is possible to preferentially restrict the number of hybridization events occurring on mismatched (methylated) target but at the expense of picking only a limited number of matched (un-methylated) targets.

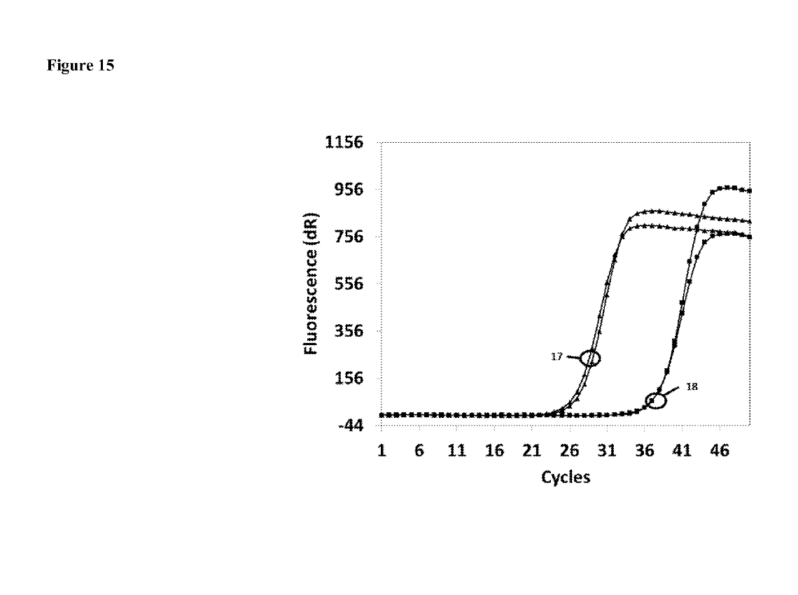

FIG. 15 shows that increasing the number of linear amplification cycles for the LATE-PCR SuperSelective limiting primer from one to ten allows better amplification of the matched (unmethylated) targets (Curves 17) but enough mismatched (methylated) targets hybridize under these conditions to allow amplification of all three replicates (Curves 18). These experiments demonstrate that LEL PCR allows for more stringent amplification of SuperSelective primers and fewer number of cycles where the SuperSelective primers interrogate the mismatched targets but that these changes do not improve the amplification selectivity. These experiments also demonstrate that SuperSelective primers can be targeted to sequence differences resulting from bisulfite conversion of methylation-dependent differences in a pair of targets.

### Example 4

### LATE-PCR SuperSelective Primers in Combination with a Temperature-Dependent Reagent

The experiments in Example 3 were performed in the presence of another version of a Temperature-Dependent Reagent (Reagent 2, a double-stranded DNA with terminal modifications that include a fluorophore and a quencher, described in U.S. Patent application publication 2012/0088275) to test for improvements in selectivity.

Fluorescent Temperature Dependent Reagent 2:
[SEQ 12]
5′ QSR670-CAGCTGCACTGGGAAGGGTGCAGTCTGACC-C3 3′
[SEQ 13]
5′ GGTCAGACTGCACCCTTCCCAGTGCAGCTG-BHQ2 3′

Amplifications were carried out in replicate reactions in a Stratagene MX3005P thermal cycler (Agilent, Santa Clara, Calif.). PCR assays were performed in a 15 μl volume consisting of 1× Platinum Taq buffer (Invitrogen, Carlsbad, Calif.), 3 mM MgCl2, 250 μM of each deoxynucleotide triphosphate, 50 nM of LATE-PCR SuperSelective primer, 1000 nM for LATE-PCR reverse primer, 0.24×SYBR Green (Invitrogen, Carlsbad, Calif.), 1.5 units of Platinum Taq DNA polymerase (Invitrogen, Carlsbad, Calif.), 25 nM Reagent 2 and 10,000 copies of either matched un-methylated target or matched methylated target. The reactions were first incubated at 95° C. for three minutes, followed by 10 cycles of denaturation at 95° C. for 15 seconds and primer annealing at 70° C. for 15 seconds, and primer extension at 80° C. for 30 seconds (note: primer extension was done at 80° C. instead of the customary 72° C.-75° C. to prevent further hybridization events of the SuperSelective primer during extension), one cycle of denaturation at 95° C. for 15 seconds and primer annealing at 60° C. for 15 seconds, and primer extension at 72° C. for 30 seconds, 50 cycles of 95° C. for 15 seconds and primer annealing/extension at 75° C. for 30 seconds, and a melting from 25° C. to 95° C. with fluorescent acquisition in the Cal Orange channel (to monitor the fluorescence of Reagent 2). SYBR Green fluorescent intensity was measured during each extension step throughout the course of each reaction to follow the amplification reaction in real time.

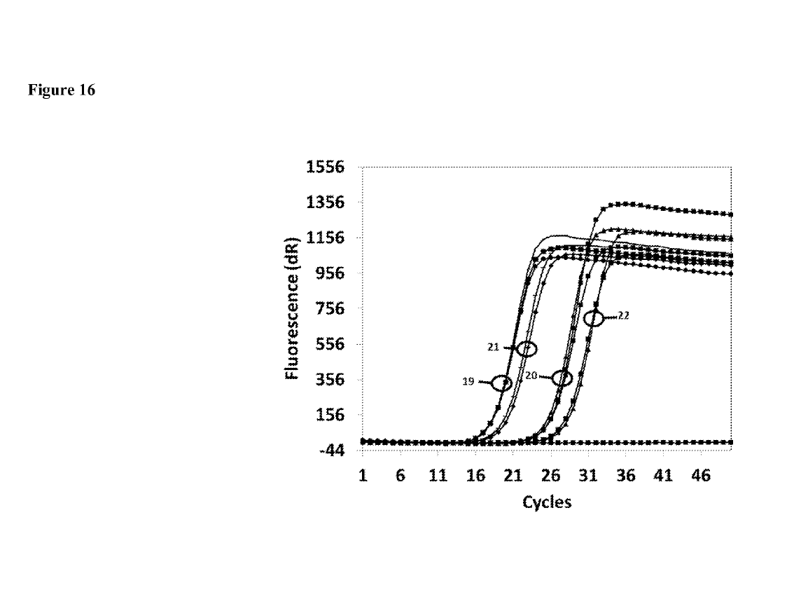

FIG. 16 shows that addition of 25 nM of the Reagent 2 increased the selectivity of the LATE-PCR SuperSelective primers by 0.8 Ct values. The delta Ct value between the matched unmethylated target (Curves 19) and the mismatched methylated target (Curves 20) was 7.3 cycles compared to the delta Ct value between the matched unmethylated target +25 nM Reagent 2 (Curves 21) and the mismatched methylated target+25 nM Reagent 2 (Curves 22).

Reagent 2 can be optimized further to achieve improved selectivity similar to Temperature-Dependent Reagent EP003, as described in provisional patent application No. 61/755,872, which is hereby incorporated by reference. Reagent 2 can be combined with other versions of Temperature Dependent Reagents to achieve even more improvements in selectivity, as described in U.S. patent application Ser. No. 13/256,038, which is hereby incorporated by reference.

These results, along with those in Example 2, demonstrate that Temperature Dependent Reagents can be used instead of precise temperature regulation to control the specificity of LATE-PCR SuperSelective primers. These examples demonstrate that LATE-PCR SuperSelective primers can be optimized by adjusting thermal cycles and by adjusting the type and combinations of Temperature Dependent Reagents such as EP003 and Reagent 2. Further possible optimizations to achieve maximal allele discrimination with SuperSelective primer include adjusting length of the foot, the length and melting temperature of the bridge sequence, the melting temperature of the anchor sequence of the SuperSelective primers themselves. Since the selective steps for differential amplification take place in the in the very early cycles, multiplexing with different pairs of SuperSelective limiting primers and reverse excess primers will reflect the number of starting copies of different alleles are present in the initial sample.

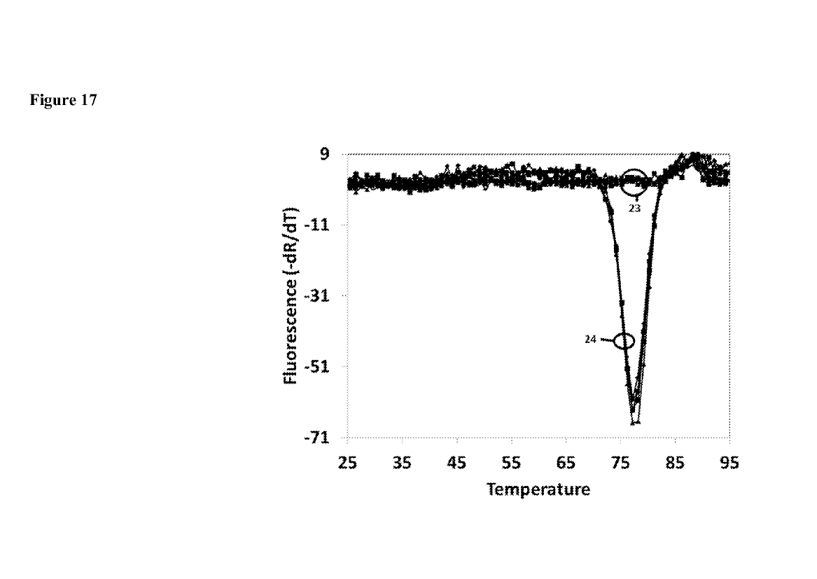

FIG. 17 shows that Reagent 2 present in the samples from FIG. 16 can be readily visualized by virtue of its own fluorescence (Cal Orange, in this particular example). Curves 23 correspond to reactions without Reagent 2; Curves 24 corresponds to reactions with 25 nM Reagent 2.

### Example 5

### Sites of Methylation/Demethylation Selectively Amplified Using LEL-PCR and Visualized Using Fluorescent Probes

Selective amplification of targets based on the methylation status of a specific site allows for the determination of the methylation status of sites internal to the amplified region. Such internal methylation differences are converted to sequence differences after bisulfite treatment. To model this situation, the experiment depicted FIG. 15 and in Example 3 was repeated using a pair of matched unmethylated targets, one simulating an internal un-methylated CpG after bisulfite conversion within the amplified region ([SEQ 10], used in Example 3) and the other simulating the sequence after bisulfite conversion corresponding to the same site if it were methylated. These internal sequence differences were visualized using a fluorescent single mismatched tolerant fluorescent probe.

The sequence of the targets used was as follows:

Matched simulated unmethylated target with an
internal unmethylated site:
[SEQ 10]
5′CTAAAGCCACCTCCTTACTTTGCCTCCTTCTGCA [symbol] GGTATTCTTTC
TCTTCCGCATGCACCAGTTTGGCCTGCCCAAAATCTGTGATCTTGACAT
GCTGCGGTGTTTTCACCAGTACGTTCCTGG3′
Matched simulated unmethylated target with an
internal methylated site:
[SEQ 14]
5′CTAAAGCCACCTCCTTACTTTGCCTCCTTCTGCA [symbol] GGTATTCTTTC
TCTTCCGCATGCACCAGTTTGGCCTGCCCAAAATCTGTGATCTTGACAT
GCTGCGGTGTTTTCACCAGTACGTTCCTGG3′

The simulated unmethylated site within the matched unmutilated target is indicated below in bold and italics. The simulated unmethylated site used for selective amplification is shown underlined and in bold.

Methylation mismatch-tolerant fluorescent probe:
[SEQ 15]
Quasar 670 5′ CCAAACTGGTGCGGG 3′ BHQ 2

The underline nucleotide in bold is matched to the internal methylated site and mismatched to the internal un-methylated site. The predicted Tm of the probe-amplicon hybrids for the unmethylated and methylated internal site calculated using Visual OMP (DNA Software, Ann Arbor, Mich.) were 47° C. and 57° C.

Amplifications were carried out in replicate reactions in a Stratagene MX3005P thermal cycler (Agilent, Santa Clara, Calif.). PCR assays were performed in a 15 μl volume consisting of 1× Platinum Taq buffer (Invitrogen, Carlsbad, Calif.), 3 mM MgCl2, 250 μM of each deoxynucleotide triphosphate, 50 nM of LATE-PCR SuperSelective primer, 1000 nM for LATE-PCR reverse primer, 0.24×SYBR Green (Invitrogen, Carlsbad, Calif.), 1.5 units of Platinum Taq DNA polymerase (Invitrogen, Carlsbad, Calif.), 25 nM Reagent 2 and 10,000 copies of either matched un-methylated target with an internal methylated site or matched un-methylated target with an internal unmethylated site. The reactions were first incubated at 95° for three minutes, followed by 10 cycles of denaturation at 95° C. for 15 seconds and primer annealing at 70° C. for 15 seconds, and primer extension at 80° C. for 30 seconds (note: primer extension was done at 80° C. instead of the customary 72° C.-75° C. to prevent further hybridization events of the SuperSelective primer during extension), one cycle of denaturation at 95° C. for 15 seconds and primer annealing at 60° C. for 15 seconds, and primer extension at 72° C. for 30 seconds, 50 cycles of 95° C. for 15 seconds and primer annealing/extension at 75° C. for 30 seconds, and a melting from 25 C to 95° C. with fluorescent acquisition in the Cal Orange channel (to monitor the fluorescence of Reagent 2) and in the Quasar 670 channel to monitor probe fluorescence. SYBR Green fluorescent intensity was measured during each extension step throughout the course of each reaction to follow the amplification reaction in real time

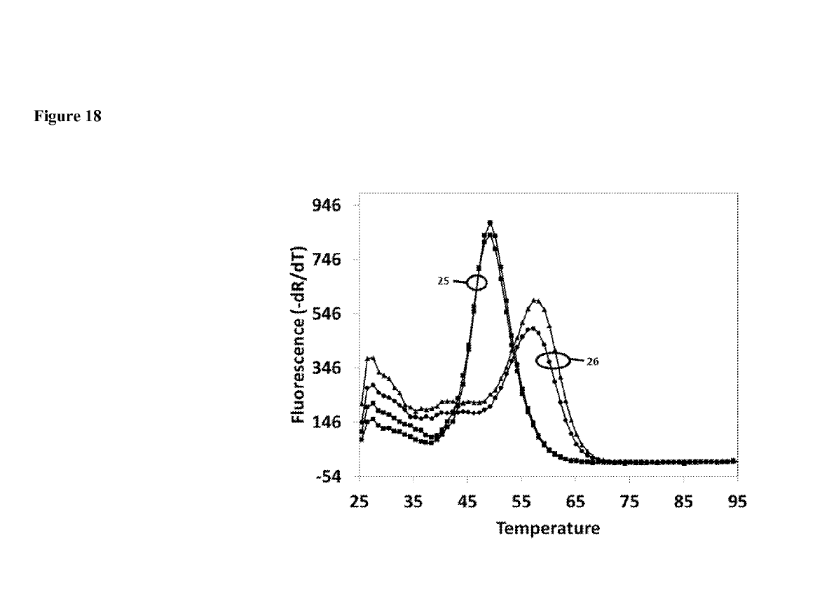

FIG. 18 shows that the probe readily distinguished selectively amplified amplicons containing a simulated internal unmethylated site from those containing the same simulated site but methylated. Curves 25 correspond to amplicons with an internal unmethylated site, Curves 26 correspond to amplicons with an internal methylated site.

### Example 6

### Temperature Dependent Reagents to Control the Activity of the Polymerase within a Range of Temperatures

Examples 2 and 4 above illustrate the use of Temperature-Dependent Reagents EP003 and Reagent 2 to control the specificity of Taq DNA polymerase within a range of temperatures where these reagents remain double-stranded simply by changing the concentration of the reagent. This example demonstrates the use of another type of Temperature-Dependent Reagents, Hairpin Reagent 1, a double-dabcyl hairpin oligonucleotide, to control the activity of the Taq DNA polymerase. Hairpin Reagent 1 (described in U.S. Pat. No. 7,517,977, included by reference herein in its entirety) comprising a hairpin oligonucleotide having a stem duplex greater than six nucleotides in length and a stabilized stem terminus by 5′ and 3′ terminal dabcyl modifications.

HairpinReagent 1:
[SEQ 16]
5′ [5-DABCYL] GAATAATATAG-loop sequence-
CTATATTATTC Q] 3′

The melting temperature of the stem duplex of Hairpin Reagent 1 is 53° C.

The methylated and unmethylated targets used in Example 3 [SEQ 10 and SEQ 11] were amplified with Forward EGFR anchor primer [SEQ 5—Example 1] and Reverse EGFR primer [SEQ 2—Example 1] in the presence of recombinant Taq DNA polymerase supplemented with either Taq antibody or with 1 μM Hairpin Reagent 1 to test whether Hairpin Reagent 1 prevents amplification of these targets during temperature cycling despite these targets being at a starting copy number of 1,000,000 each.

Amplifications were carried out in replicate reactions in a Stratagene MX3005P thermal cycler (Agilent, Santa Clara, Calif.). PCR assays were performed in a 30 μl volume consisting of 1× Platinum Taq buffer (Invitrogen, Carlsbad, Calif.), 3 mM MgCl2, 250 μM of each deoxynucleotide triphosphate, 60 nM Forward EGFR Anchor primer, 60 nM for Reverse EGFR primer, 0.24×SYBR Green (Invitrogen, Carlsbad, Calif.), 1.5 units of Taq DNA polymerase (Invitrogen, Carlsbad, Calif.), 1,000,000 copies of either matched un-methylated target with an internal methylated site or matched un-methylated target with an internal unmethylated site and either 1.5 units of Taq DNA antibody (Invitrogen, Carlsbad, Calif.) or 1 μM Hairpin Reagent 1 (Biosearch, Petaluma, Calif.). The reactions were first incubated at 95° C. for three minutes, followed by 50 cycles of denaturation at 95° C. for 15 seconds and primer annealing at 60° C. for 15 seconds, and primer extension at 72° C. for 30 seconds. SYBR Green fluorescent intensity was measured during each extension step throughout the course of each reaction to follow the amplification reaction in real time.

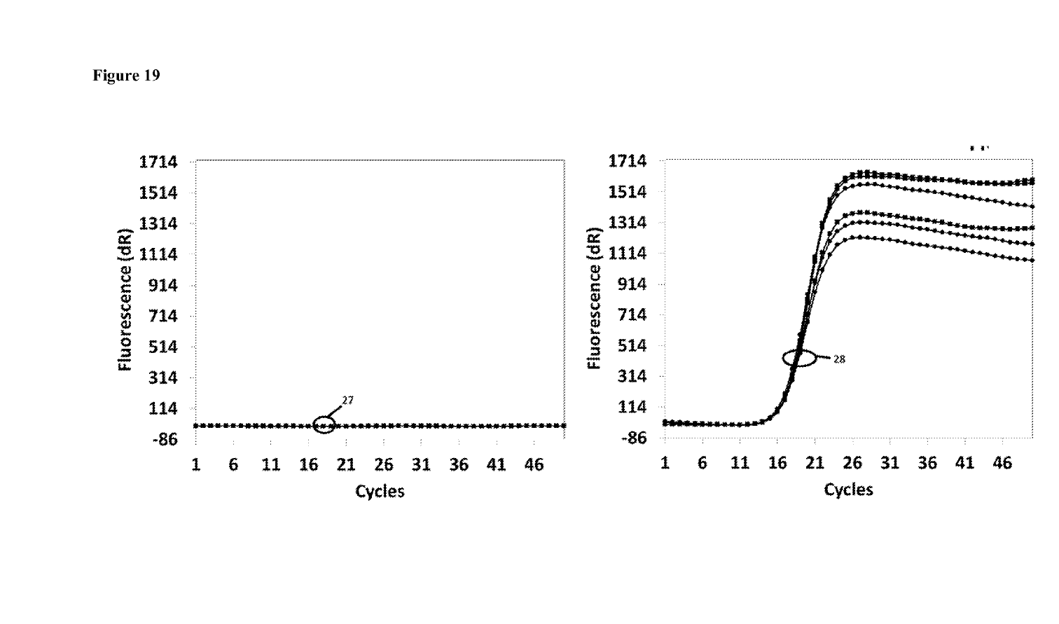

FIG. 19 shows that Hairpin Reagent 1 (Curve 27) successfully prevented amplification over a range of temperature. Control reactions with Taq DNA polymerase and Taq DNA polymerase antibody demonstrate that failure to amplify was due to Hairpin Reagent 1 controlling the activity of Taq DNA polymerase at the annealing temperature (Curve 28). This experiment demonstrate that Temperature-Dependent Reagents rather than precise temperature control can be used to define the activity of Taq DNA polymerase during PCR amplification.

### Example 7

### Temperature Imprecise PCR (TI-PCR)

A TI-PCR reaction is carried out using LEL-PCR amplification reaction and the SuperSelective Limiting Primer and the 5′-extended Excess Primer and the temperature and cycling conditions described in Example 3. The reaction mixture is optimized by combining 300-1000 nM of a double-dabcylated hairpin oligonucleotide with either 12.5-200 nM of a three dabcyl double-stranded oligonucleotide, such as EP003 (Example 2) or 12.5-200 nM of a double-stranded oligonucleotide labeled with a fluorophore and a quencher, as in Example 4. The double-hairpin oligonucleotide serves as a “hot start-like” inhibitor of polymerase activity prior to amplification and at any time during amplification when the temperature of the reaction vessel falls into Zone 3 of FIG. 20, i.e. approximately 2° C. or more below the temperature needed for hybridization and extension of the Excess primer at its initial low-Tm. The concentration and melting temperatures of the three dabcl double-stranded oligonucleotide or the double-stranded fluorophore/quencher oligonucleotide are optimized to increase the specificity of the DNA polymerase in Zone 2 of FIG. 20. As the two strands of these double-stranded oligonucleotides begin to hybridize at the highest temperatures of Zone 2, the effective double-stranded concentrations of these reactions increase as the temperature of Zone 2 decreases. For this reason, a moderate-Tm primer or a high Tm primer, such as a SuperSelective Primer before and after incorporation, can rapidly bind to and extend on its target as the temperature of the reaction falls into Zone 2. In contrast, a low-Tm primer, such as the Excess primer prior to incorporation, requires a lower temperature and longer time to bind to and extend on its template strand. As illustrated in FIG. 20, this is accomplished by delaying the heat pulse for one cycle. However, in contrast to Example 3, the length of this tow temperature step does not need to be precisely controlled in TI-PCR because the activity of the enzyme is inhibited as long as the reaction is in Zone 3. As depicted in FIG. 20, when heat is pulsed back into the system, the total time spent in the lower range of Zone 2 is sufficiently long to allow for initial hybridization and extension of the Excess Primer. After this thermal cycle the complementary sequences of the Limiting (SuperSelective Primer) the 5′-extended Excess Primer is present in the amplicon strands. For this reason, both primers become high-Tm primers which can hybridize and extend rapidly when the reaction enters Zone 2. Thus, the high frequency heat pulse can now be used to exponentially amplify both strands until the Limiting Primer runs out. Thereafter, high frequency heat pulses can continue to be used for linear amplification of just the Excess Primer Strand.

The reaction is paused, or completed by delaying the pulsation of heat at the desired cycle. The temperature of the reaction decreases at a rate that depends on the ambient temperature. As before, the activity of the DNA polymerase is inhibited by the double-dabcyl hairpin oligonucleotide as soon as Zone 3 is reached. One or more low-Tm double labeled fluorescent probes, or quencher only probes that have been present throughout the reaction bind to the accumulated single-strands and generate a characteristic signal. If a double-strand DNA binding dye is also present in the reaction, it too binds the double-stranded amplicon molecules and the single-stranded amplicon molecules having bound probes. These closed-tube methods of amplicon analysis are described U.S. patent application publication numbers: 2012/0282611 and 2013/0095479, each of which is hereby incorporated by reference in its entirety.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

1. A method of detecting the methylation status of a CpG dinucleotide located in a target region of a DNA molecule, the method comprising the steps of:
a) contacting the DNA molecule with a solution comprising bisulfate under conditions such that a majority of unmethylated cytosine residues in the DNA molecule are converted to uracil and a majority of methylated cytosine residues in the DNA molecule remain cytosine;
b) amplifying the target region of the bisulfite-treated DNA molecule using LATE-PCR or LEL-PCR to generate an amplification product;
c) determining whether the CpG dinucleotide was converted to uracil in step a).

2. The method of claim 1, wherein LATE-PCR is used in step b).

3. The method of claim 1, wherein LEL-PCR is used in step b).

4. The method of claim 1, wherein TI-PCR is used to perform the LATE-PCR or LEL-PCR amplification.

5. The method of claim 1, wherein one or more SuperSelective primers are used during the amplification process of step b).

6. The method of claim 1, wherein the target region encompasses a CpG island.

7. The method of claim 1, wherein step c) comprises hybridizing the amplification product to a set of Lights-On/Lights-Off probes or to a Lights-Off Only probe and a dsDNA fluorescent dye.

8. The method of claim 6, wherein a Lights-On probe hybridizes to the amplification product at the position of a CpG dinucleotide.

9. The method of claim 1, wherein step c) comprises sequencing at least part of the amplification product.

10. The method of claim 9, wherein the sequencing is performed using an assay selected from the group consisting of chain termination sequencing, sequencing by ligation, sequencing by synthesis, pyrosequencing, ion semiconductor sequencing, single-molecule real-time sequencing, dilute-‘n’-go sequencing and 454 sequencing.

11. The method of claim 10, wherein the sequencing step comprises dilute-‘n’-go sequencing.

12. The method of claim 1, wherein the methylation status of more than one CpG dinucleotide located in the target region is detected.

13. The method of claim 12, wherein step c) comprises hybridizing the amplification product to a first set of Lights-On/Lights-Off probes and a second set of Lights-On/Lights-Off probes.

14. The method of claim 13, wherein the first set of Lights-On/Lights-Off probes and the second set of Lights-On/Lights-Off probes are tagged with the same fluorescent label.

15. The method of claim 13, wherein the first set of Lights-On/Lights-Off probes and the second set of Lights-On/Lights-Off probes are tagged with different fluorescent labels.

16. The method of claim 1, further comprising the step of purifying the DNA molecule such that it is substantially protein-free prior to step a).

17. The method of claim 16, wherein the purification step is performed using a proteinase K lysis solution containing 10 mM Tris-Cl pH 8.3, 5 μM SDS, and 0.1 mg/ml Proteinase K.

18. The method of claim 1, wherein the DNA molecule is fragmented prior to step a).

19. The method of claim 18, where the DNA is fragmented using a restriction enzyme that cuts the DNA molecule outside of the region amplified in step b).

20. The method of claim 1, wherein at least 90% of unmethylated cytosine residues in the DNA molecule are converted to uracil during step a) and less than 10% of methylated cytosine residues are converted to uracil during step a).

21. A method of detecting the methylation status of a CpG dinucleotide located in a target region, the method comprising the steps of:
a) dividing a genomic DNA sample into a control genomic DNA solution containing a control DNA molecule containing the target region from a test genomic DNA solution containing a test DNA molecule containing the target region;
b) contacting the test DNA molecule with a solution comprising bisulfite under conditions such that a majority of unmethylated cytosine residues in the DNA molecule are converted to uracil and a majority of methylated cytosine residues in the DNA molecule remain cytosine;
c) amplifying the target region of the test DNA molecule using LATE-PCR or LEL-PCR using a first primer set complementary to bisulfite treated DNA to generate a first amplification product;
d) amplifying the target region of the control DNA molecule using LATE-PCR or LEL PCR using a second primer set complementary to native DNA to generate a second amplification product;
e) incubating the first amplification product with a first set of Lights-On/Lights-Off probes or with a Lights-Off Only probe and a dsDNA fluorescent dye at different temperatures to generate a first fluorescent signature;
f) incubating the second amplification product with a second set of Lights-On/Lights-Off probes or with a second Lights-Off Only probe and a dsDNA fluorescent dye to generate a second fluorescent signature; and
g) comparing the first fluorescent signature with the second fluorescent signature to determine whether the CpG dinucleotide was methylated.

22. The method of claim 21, wherein LATE-PCR is used in steps c) and d).

23. The method of claim 21, wherein LEL-PCR is used in steps c) and d).

24. The method of claim 21, wherein TI-PCR is used to perform the LATE-PCR or LEL-PCR amplification.

25. The method of claim 21, wherein one or more SuperSelective primers are used during the amplification process of steps c) and d).

26. The method of claim 21, wherein the target region encompasses a CpG island.

27. The method of claim 21, wherein the methylation status of more than one CpG dinucleotide located in the target region is detected.

28. The method of claim 21, further comprising the step of purifying the DNA molecule such that it is substantially protein-free prior to step a).

29. The method of claim 28, wherein the purification step is performed using a proteinase K lysis solution containing 10 mM Tris-Cl pH 8.3, 5 μM SDS, and 0.1 mg/ml Proteinase K.

30. The method of claim 21, wherein the DNA molecules are fragmented prior to step a).

31. The method of claim 30, where the DNA molecules are fragmented using a restriction enzyme that cuts the DNA molecule outside of the region amplified in step b).

32. The method of claim 21, wherein at least 90% of unmethylated cytosine residues in the DNA molecule are converted to uracil during step b) and less than 10% of methylated cytosine residues are converted to uracil during step a).
